(19) United States
(12) Patent Application Publication
(10) Pub. No.: US 2019/0151456 A1
(43) Pub. Date: May 23, 2019

(54) Compositions of Engineered Exosomes and Methods of Loading Luminal Exosome Payloads

(71) Applicant: Codiak BioSciences, Inc., Cambridge, MA (US)

(72) Inventors: Russell E. McConnell, Somerville, MA (US); Kevin P. Dooley, Boston, MA (US); Rane A. Harrison, Belmont, MA (US); Ke Xu, Sudury, MA (US); Damian J. Houde, Plymouth, MA (US); Sonya Haupt, Cambridge, MA (US); John D. Kulman, Belmont, MA (US); Douglas E. Williams, Boston, MA (US); Madeleine Youniss, Cambridge, MA (US)

(21) Appl. No.: 16/194,230

(22) Filed: Nov. 16, 2018

Related U.S. Application Data
(60) Provisional application No. 62/587,767, filed on Nov. 17, 2017.
(60) Provisional application No. 62/634,750, filed on Feb. 23, 2018.

(51) Int. Cl. A61K 47/42

(52) U.S. Cl. CPC A61K 47/42

(57) ABSTRACT

The present invention relates to methods of preparing a therapeutic exosome using proteins newly identified to be enriched in the lumen of exosomes. Specifically, the present invention provides methods of localizing a therapeutic peptide or protein in exosomes. The methods involve generation of lumen-engineered exosomes that include one or more of the exosome proteins at higher concentrations, a modification or a fragment of the exosome protein, or a fusion protein of the exosome protein and a therapeutic or cargo protein.

### CROSS REFERENCE TO RELATED APPLICATIONS

This application claims the benefit of U.S. Provisional Patent Application 62/587,767 filed Nov. 17, 2017, and U.S. Provisional Patent Application 62/634,750, filed Feb. 23, 2018, the disclosures of which are hereby incorporated in their entirety for all purposes.

### SEQUENCE LISTING

The instant application includes a Sequence Listing which has been submitted electronically in ASCII format and is hereby incorporated by reference in its entirety. Said ASCII copy, created on Nov. 15, 2018, is named 41406US_CRF_sequencelisting.txt and is 57,837 bytes in size.

### BACKGROUND

Exosomes are important mediators of intercellular communication. They are also important biomarkers in the diagnosis and prognosis of many diseases, such as cancer. As drug delivery vehicles, exosomes offer many advantages over traditional drug delivery methods as a new treatment modality in many therapeutic areas.

A central feature of exosomes is their ability to contain biologically active payload within their interior space, or lumen. It is well known that exosomes contain endogenous payload including mRNA, miRNA, DNA, proteins, carbohydrates, and lipids, but the ability to direct specific loading of desired therapeutic payload is currently limited. Exosomes may be loaded by overexpressing desired therapeutic payloads in a producer cell, but this loading is often of limited efficiency due to stochastic localization of the payload to cellular exosome processing centers. Alternatively, purified exosomes may be loaded ex vivo by, for example, electroporation. These methods may suffer from low efficiency or be limited to small payloads, such as siRNAs. Therefore, suitable methods for generating highly efficient and well-defined loaded exosomes are needed to better enable therapeutic use and other applications of exosome-based technologies.

### SUMMARY

An aspect of the present invention relates to novel methods of loading exosomes for therapeutic use. Specifically, the methods use protein markers that are newly identified from the lumen of exosomes. In particular, a group of proteins (e.g., myristoylated alanine rich Protein Kinase C substrate (MARCKS); myristoylated alanine rich Protein Kinase C substrate like 1 (MARCKSL1); and brain acid soluble protein 1 (BASP1)) were identified to be highly enriched in the lumen of exosomes. Furthermore, a short sequence of the amino terminus of BASP1 was shown to be sufficient to direct high efficiency loading of fluorescent protein cargo molecules to the same extent as the full length BASP1 protein. This fragment, which is less than ten amino acids, presents a significant advance in the field of engineered exosome loading, and allows for the efficient, reproducible loading of any therapeutic protein cargo into the lumen of exosomes with no additional steps of ex vivo manipulation. The loading of exosomes using the fusion proteins described herein produces engineered exosomes with significantly higher levels of cargo compared to any other genetic engineering method described thus far.

The proteins and peptide sequences newly identified from exosomes are used in various embodiments of the present invention. For example, some embodiments relate to generating a fusion protein by conjugating the exosome protein or protein fragment and a therapeutically relevant protein and producing an exosome containing the fusion protein in the lumen of the exosome. The native full-length protein or a biologically active fragment of the therapeutically relevant protein can be transported to the lumen of exosomes by being conjugated to the exosome-enriched proteins or protein fragments.

The present invention further relates to generation or use of a lumen-engineered exosome designed for more efficient loading, or for loading of a therapeutically relevant protein in the lumen of an exosome. For example, the exosome lumen can be modified to contain a higher concentration of the native full-length exosome protein and/or a fragment or a modified protein of the native exosome protein in the lumen.

Some embodiments of the present invention relate to a producer cell or a method of generating the producer cell for producing such a lumen-engineered exosome. An exogenous polynucleotide can be introduced transiently or stably into a producer cell to generate a lumen-engineered exosome from the producer cell.

In one aspect, provided herein are exosomes comprising a target protein, wherein at least a part of the target protein is expressed from an exogenous sequence, and the target protein comprises BASP1 or a fragment thereof.

In some embodiments, the target protein is present in the lumen of the exosome at a higher density than a different target protein of a different exosome, wherein the different target protein comprises a conventional exosome protein or a variant thereof. In some embodiments, the conventional exosome protein is selected from the group consisting of CD9, CD63, CD81, PDGFR, GPI anchor proteins, lactadherin, LAMP2, LAMP2B, and a fragment thereof.

In some embodiments, the target protein comprises a polypeptide of any of SEQ ID NOs: 3-15. In some embodiments, wherein the target protein comprises a polypeptide of SEQ ID NO: 14.

In some embodiments, the exosome is produced from a cell genetically modified to comprise the exogenous sequence, optionally wherein the cell is an HEK293 cell. In some embodiments, the cell comprises a plasmid comprising the exogenous sequence. In some embodiments, the cell comprises the exogenous sequence inserted into a genome of the cell. In some embodiments, the exogenous sequence is inserted into a genomic site located 3′ or 5′ end of a genomic sequence encoding BASP1 or a fragment thereof. In some embodiments, the exogenous sequence is inserted into a genomic sequence encoding BASP1.

In some embodiments, the target protein is a fusion protein comprising BASP1 or a fragment thereof, and a therapeutic peptide. In some embodiments, the therapeutic peptide is selected from the group consisting of a natural peptide, a recombinant peptide, a synthetic peptide, or a linker to a therapeutic compound. In some embodiments, wherein the therapeutic compound is selected from the group consisting of nucleotides, amino acids, lipids, carbohydrates, and small molecules. In some embodiments, the therapeutic peptide is an antibody or a fragment thereof. In some embodiments, the therapeutic peptide is an antigen. In some embodiments, the therapeutic peptide is a component of a genome editing complex. In some embodiments, the genome editing complex is a CRISPR/Cas9 genome editing complex.

In some embodiments, the target protein is a fusion protein comprising BASP1 or a fragment thereof, and a viral capsid protein. In some embodiments, the viral capsid protein is from adeno-associated virus

In some embodiments, further comprising a second target protein, wherein the second target protein comprises PTGFRN, BSG, IGSF3, IGSF2, ITGB1, ITGA4, SLC3A2, ATP transporter, or a fragment thereof.

Accordingly, in an aspect, the present invention provides an exosome comprising a target protein, wherein at least a part of the target protein is expressed from an exogenous sequence, and the target protein comprises MARCKS, MARCKSL1, BASP1 or a fragment or a modification thereof.

In some embodiments, the target protein is present in the exosome at a higher density than a different target protein in a different exosome, wherein the different target protein comprises a conventional exosome protein or a variant thereof. In some embodiments, the conventional exosome protein is selected from the group consisting of CD9, CD63, CD81, PDGFR, GPI anchor proteins, lactadherin, LAMP2, LAMP2B, and a fragment thereof.

In some embodiments, the exosome is produced from a cell genetically modified to comprise the exogenous sequence, optionally wherein the cell is an HEK293 cell.

In some embodiments, the cell comprises a plasmid comprising the exogenous sequence.

In some embodiments, the exogenous sequence is inserted into a genomic site located 3′ or 5′ relative to a genomic sequence encoding MARCKS, MARCKSL1, or BASP1. In some embodiments, the exogenous sequence is inserted into a genomic sequence encoding MARCKS, MARCKSL1, or BASP1.

In some embodiments, the target protein is a fusion protein comprising MARCKS, MARCKSL1, BASP1, or a fragment thereof, and a therapeutic peptide.

In some embodiments, the therapeutic peptide is selected from the group consisting of a natural peptide, a recombinant peptide, a synthetic peptide, or a linker to a therapeutic compound. In some embodiments, the therapeutic compound is selected from the group consisting of nucleotides, amino acids, lipids, carbohydrates, and small molecules. In some embodiments, the therapeutic peptide is an antibody or a fragment or a modification thereof. In some embodiments, the therapeutic peptide is an enzyme, a ligand, a receptor, a transcription factor, or a fragment or a modification thereof. In some embodiments, the therapeutic peptide is an antimicrobial peptide or a fragment or a modification thereof.

In some embodiments, the exosome further comprises a second target protein, wherein the second target protein comprises MARCKS, MARCKSL1, BASP1, or a fragment thereof. In some embodiments, the exosome further comprises a second target protein, wherein the second target protein comprises PTGFRN, BSG, IGSF2, IGSF3, IGSF8, ITGB1, ITGA4, SLC3A2, ATP transporter or a fragment thereof.

In some embodiments, the target protein comprises a peptide of (M)(G)(G/A/S)(K/Q)(L/F/S/Q)(S/A)(K)(K) (SEQ ID NO: 118). In some embodiments, the target protein comprises a peptide of (M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In some embodiments, the target protein comprises a peptide of (M)(G)(π)(ξ)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu).

In some embodiments, the target protein comprises a peptide of any one of SEQ ID NO: 4-110. In some embodiments, the target protein comprises a peptide of MGXKLSKKK, wherein X is any amino acid (SEQ ID NO: 116). In some embodiments, the target protein comprises a peptide of SEQ ID NO: 110. In some embodiments, the target protein comprises the peptide of SEQ ID NO: 13.

In some embodiments, the target protein further comprises a cargo peptide.

In another aspect, the present invention provides a pharmaceutical composition comprising the exosome and an excipient.

In some embodiments, the pharmaceutical composition is substantially free of macromolecules, wherein the macromolecules are selected from nucleic acids, exogenous proteins, lipids, carbohydrates, metabolites, and a combination thereof.

In yet another aspect, the present invention provides a population of cells for producing the exosome provided herein.

In some embodiments, the population of cells comprises an exogenous sequence encoding the target protein comprising MARCKS, MARCKSL1, BASP1 or a fragment or a modification thereof. In some embodiments, the population of cells further comprise a second exogenous sequence encoding a second target protein, wherein the second target protein comprises MARCKS, MARCKSL1, BASP1 or a fragment or a modification thereof. In some embodiments, the population of cells further comprises a second exogenous sequence encoding a second target protein, wherein the second target protein comprises PTGFRN, BSG, IGSF2, IGSF3, IGSF8, ITGB1, ITGA4, SLC3A2, ATP transporter or a fragment thereof.

In some embodiments, the exogenous sequence is inserted into a genomic sequence encoding MARCKS, MARCKSL1, or BASP1, wherein the exogenous sequence and the genomic sequence encodes the target protein. In some embodiments, the exogenous sequence is in a plasmid.

In some embodiments, the exogenous sequence encodes a therapeutic peptide. In some embodiments, the therapeutic peptide is selected from a group consisting of a natural peptide, a recombinant peptide, a synthetic peptide, or a linker to a therapeutic compound. In some embodiments, the therapeutic compound is selected from the group consisting of nucleotides, amino acids, lipids, carbohydrates, and small molecules. In some embodiments, the therapeutic peptide is an antibody or a fragment or a modification thereof. In some embodiments, the therapeutic peptide is an enzyme, a ligand, a receptor, a transcription factor, or a fragment or a modification thereof. In some embodiments, the therapeutic peptide is an antimicrobial peptide or a fragment or a modification thereof.

In some embodiments, the exogenous sequence encodes a targeting moiety. In some embodiments, the targeting moiety is specific to an organ, a tissue, or a cell.

In some embodiments, the second target protein further comprises a targeting moiety. In some embodiments, the targeting moiety is specific to an organ, a tissue, or a cell.

In one aspect, the present invention provides a polypeptide for modifying an exosome, comprising a sequence of (i) (M)(G)(G/A/S)(K/Q)(L/F/S/Q)(S/A)(K)(K) (SEQ ID NO: 118); (ii) (M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu); or (iii) (M)(G)(π)(Φ/π)(S/A/G/N)(+)(+), wherein each parenthetical position represents an amino acid, and wherein π is any amino acid selected from the group consisting of (Pro, Gly, Ala, Ser), is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu).

In some embodiments, the polypeptide comprises a sequence of any of SEQ ID NO: 4-110. In some embodiments, the polypeptide comprises a sequence of SEQ ID NO: 13. In some embodiments, the polypeptide comprises a sequence of SEQ ID NO: 110. In some embodiments, the polypeptide comprises a sequence of MGXKLSKKK, wherein X is any amino acid (SEQ ID NO: 116).

In some embodiments, the polypeptide is fused to a cargo peptide. In some embodiments, the polypeptide is fused to the N-terminus of the cargo peptide.

In one aspect, the present invention provides a polynucleotide construct comprising a coding sequence encoding the polypeptide provided herein. In some embodiments, the coding sequence is codon optimized.

In another aspect, the present invention provides a method of making an engineered exosome, comprising the steps of: a. introducing into a cell a nucleic acid construct encoding a fusion polypeptide comprising (i) a first sequence encoding MARCKS, MARCKSL1, BASP1 or a fragment or a modification thereof, and (ii) a second sequence encoding a cargo peptide; b. maintaining the cell under conditions allowing the cell to express the fusion polypeptide; and c. obtaining the engineered exosome comprising the fusion polypeptide from said cell.

In some embodiments, the first sequence comprises a sequence of (i) (M)(G)(G/A/S)(K/Q)(L/F/S/Q)(S/A)(K)(K) (SEQ ID NO: 118); (ii) M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu); or (iii) (M)(G)(π)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu).

In some embodiments, the polynucleotide comprises a sequence of any of SEQ ID NO: 4-110. In some embodiments, the polynucleotide comprises a sequence of SEQ ID NO: 13. In some embodiments, the polynucleotide comprises a sequence of SEQ ID NO: 110. In some embodiments, the polynucleotide comprises a sequence of MGXKLSKKK, wherein X is any amino acid (SEQ ID NO: 116).

In some embodiments, the fusion polypeptide is present in the lumen of the engineered exosome at a higher density than a different target protein in a different exosome, wherein the different target protein comprises a conventional exosome protein or a variant thereof. In some embodiments, the fusion polypeptide is present at more than 2 fold higher density than the different target protein in the different exosome. In some embodiments, the fusion polypeptide is present at more than 4 fold, 16 fold, 100 fold, or 10,000 fold higher density than the different target protein in the different exosome.

### BRIEF DESCRIPTION OF THE DRAWINGS

The figures depict various embodiments of the present invention for purposes of illustration only. One skilled in the art will readily recognize from the following discussion that alternative embodiments of the structures and methods illustrated herein may be employed without departing from the principles of the invention described herein.

FIG. 1 provides an image of sample-containing Optiprep™ density gradient after ultracentrifugation. Marked with brackets are the top fraction containing exosomes (“Top”), the middle fraction containing cell debris (“Middle”) and the bottom fraction containing high density aggregates and cellular debris (“Bottom”).

FIG. 2 is a dot-graph showing proteins identified from the top fraction (Y-axis) and proteins identified from the bottom fraction (X-axis) of Optiprep™ ultracentrifugation. Proteins plotted above the dotted line represent exosome-enriched proteins (including MARCKS, MARCHSL1 and BASP1), while those below the dotted line represent proteins not specific to exosomes.

FIG. 3 provides a tryptic peptide coverage map of MARCKS (SEQ ID NO: 1).

FIG. 4 provides a tryptic peptide coverage map of MARCKSL1 (SEQ ID NO: 2).

FIG. 5 provides a tryptic peptide coverage map of BASP1 (SEQ ID NO: 3).

FIG. 6A shows a picture from protein blotting of total cell lysate (left) and purified exosome populations (right) collected from HEK293 cells. Western blotting of the gel provided in FIG. 6A shows that MARCKS (FIG. 6B), MARCKSL1 (FIG. 6C), and BASP1 (FIG. 6D) are localized in purified exosomes and either not detected in total cell lysate or are at substantially lower levels in cell lysate as compared to exosomes.

FIG. 7 shows the fluorescence intensity of purified exosomes containing GFP fused to a fragment of MARCKS containing amino acids 1-30, CD81, or pDisplay.

FIG. 8 shows the fluorescence intensity of purified exosomes containing GFP fused to full length MARCKSL1, a fragment of MARCKSL1 containing amino acids 1-30, CD81, or pDisplay.

FIG. 9 shows the fluorescence intensity of purified exosomes containing GFP fused to full length BASP1, a fragment of BASP1 containing amino acids 1-30, CD81, or pDisplay.

FIG. 10 shows a schematic of fusion proteins used to determine the minimal BASP1 N-terminal sequence that is sufficient for loading exosomes (SEQ ID NOS 122-134, respectively, in order of appearance). The fusion proteins are assigned with a number as provided under “pCB.”

FIG. 11 shows a graph from nano-flow cytometry measuring the fluorescence signal of exosomes engineered to express BASP1 fragments fused to GFP. The x-axis is numbered according to numbers assigned to various fusion proteins as provided in FIG. 10.

FIG. 12 shows a picture of a stained protein gel indicating equal loading of exosomes loaded with BASP1 fragments fused to GFP. The dotted arrow indicates the migration position of BASP1 fusion proteins. Lanes are numbered according to numbers assigned to various fusion proteins as provided in FIG. 10.

FIG. 13 shows a picture of a protein gel stained with Coomassie blue to label total protein. The dotted arrow indicates the migration position of BASP1 fusion proteins. Lanes are labeled with the numbers assigned to various fusion proteins as provided in FIG. 10.

FIG. 14 shows a picture from an anti-FLAG protein blot of purified exosomes containing BASP1 fragments fused to FLAG and GFP. Lanes are numbered according to numbers assigned to various fusion proteins as provided in FIG. 10.

FIG. 15 shows a picture from an anti-Alix protein blot of purified exosomes containing BASP1 fragments fused to FLAG and GFP, confirming equal protein loading. Lanes are numbered according to the protein sequences shown in FIG. 10.

FIG. 16A shows sequences of fusion proteins comprising a BASP1 fragment fused to a FLAG tag and GFP (SEQ ID NOS 135-142, respectively, in order of appearance). FIG. 16B shows the anti-FLAG Western blot results for exosomes purified from cells stably expressing one of the fusion proteins in FIG. 16A.

FIG. 17A shows sequences from a BASP1 fragment (1-30) (SEQ ID NO: 4) and its modifications (1-30-S6D, 1-30-S6A, and 1-30-L5Q) fused to a FLAG tag and GFP (SEQ ID NOS 143-145, respectively, in order of appearance). FIG. 17B shows the anti-FLAG Western blot results for exosomes purified from cells stably expressing one of the fusion proteins in FIG. 17A.

FIG. 18 shows an image of a Coommassie stained protein gel with exosome samples purified from cells stably expressing full-length MARCKSL1, BASP1, or amino acids 1-30 of MARCKS, MARCKSL1, or BASP1, all fused to FALG-GFP. Black arrows on the image indicate bands corresponding to the fusion proteins.

FIG. 19 shows a protein sequence alignment between the first 28 amino acids of BASP1 (conserved region 1), amino acids 1-7 and 152-173 of MARCKS (conserved region 2), and amino acids 1-7 and 87-110 of MARCKSL1 (conserved region 3).

FIG. 20A shows sequences of amino acids 1-30 of BASP1 (“BASP1-30”) (SEQ ID NO: 4) and fusion proteins comprising amino acids 1-3 of MARCKS or its modification fused to the PSD domain of MARCKS or its modification (“MARCKS-MG-PSD”, “MARCKS-MA-PSD”, “MARCKS-MG-PSD-K6S” and “MARCKS-MG-PSD-K6A”) (SEQ ID NOS 146-149, respectively, in order of appearance). Point mutations introduced into the MARCKS sequences are bolded. FIG. 20B shows anti-FLAG Western blotting results of purified exosomes from cells stably expressing the fusion proteins comprising the amino acid sequences of FIG. 20A and FLAG.

FIG. 21 shows three different consensus sequences derived from functional studies of MARCKS, MARCKSL1, and BASP1, and the amino acid requirements of each of the sequences for loading cargo into the lumen of exosomes (SEQ ID NO: 118).

FIG. 22A shows total protein (top) and an anti-Cas9 Western blot (bottom) of native exosomes or exosomes purified from cells stably expressing Cas9 fused to amino acids 1-10 or 1-30 of BASP1, as well as decreasing amounts of recombinant Cas9. FIG. 22B (top) shows a standard curve derived from Cas9 densitometry of the Western blotting results of FIG. 22A.

FIG. 22B (bottom) further provides amounts of Cas9 loaded per each purified exosome as fusion proteins conjugated to 1-30 amino acids or 1-10 amino acids fragments of BASP1, estimated based on the standard curve.

FIG. 23A shows protein gel images of exosomes purified from cells stably transfected with a construct expressing BASP1 N-terminal (amino acids 1-10) fusion to ovalbumin (“BASP1 (1-10)-OVA”) or cells stably transfected with two constructs, one expressing BASP1 N-terminal (amino acids 1-10) fusion to ovalbumin, and the other expressing CD40L fused to a transmembrane protein PTGFRN (“BASP1 (1-10)-OVA; 3XCD40L-PTGFRN”). FIG. 23A further shows an image of the protein gel loading decreasing amounts of recombinant OVA.

FIG. 23B shows anti-Ovalbumin Western blot results of the samples from FIG. 23A.

FIG. 24A shows the sequence of a camelid nanobody directed against GFP fused to amino acids 1-10 of BASP1 and a FLAG tag (SEQ ID NO: 150). FIG. 24B shows a protein gel and an anti-FLAG Western blotting results of purified exosomes from cells stably expressing the fusion protein of FIG. 24A (“BASP1(1-10)-Nanobody”) or the protein lacking the BASP1 sequence (“Nanobody”).

FIG. 25 shows a schematic of an exosome mRNA loading system comprising (i) BASP1 (1-30) fused to FLAG and monomeric or dimeric MCP variants (1XMCP(V29I) (“815”; SEQ ID NO: 111), 1XMCP (V29I/N55K) (“817”; SEQ ID NO: 112), 2XMCP(V29I) (“819”; SEQ ID NO: 113) or 2XMCP(V29I/N55K)) (“821”; SEQ ID NO: 114) and (ii) a luciferase mRNA containing 3×MS2 hairpin loops (“Luciferase-MS2 mRNA” or “811”; SEQ ID NO: 115).

FIG. 26A shows a protein gel of the exosomes containing the mRNA loading constructs described in FIG. 25, a luciferase mRNA (811) in combination with various BASP1 fusion proteins (815, 817, or 819). FIG. 26B shows an anti-FLAG Western blot of the samples in FIG. 26A.

FIG. 27A shows RT-qPCR results for the amount of Luciferase mRNA in cells (top) or exosomes (bottom) containing the mRNA loading constructs shown in FIG. 25. FIG. 27B shows a table quantitating the amount of Luciferase mRNA in purified exosomes from the samples in FIG. 27A, including fold-enrichment from stochastic loading of Luciferase mRNA.

FIG. 28 shows schematic diagrams of CD40L trimers fused to N-terminal fragments of MARCKS, MARCKSL1, and BASP1 to allow for external surface display of transmembrane proteins anchored in the exosome lumen.

FIG. 29A shows the results of mouse B-cell activation in cultures incubated with CD40L surface expression exosomes fused to N-terminal fragments of MARCKS, MARCKSL1, and BASP1. FIG. 29B shows the results of human B-cell activation in cultures incubated with CD40L surface expression exosomes fused to N-terminal fragments of MARCKS, MARCKSL1, and BASP1. FIG. 29C shows a chart of relative potency for different CD40L surface display exosomes when fused to N-terminal sequences of MARCKS, MARCKSL1, BASP1, or full-length PTGFRN.

FIG. 30 shows the number of peptide spectrum matches (PSMs) of luminal proteins (MARCKS, MARCKSL1, and BASP 1) and conventional exosome proteins (CD81 and CD9) in exosomes purified from various cell lines of different origins (HEK293SF, kidney; HT1080, connective tissue; K562, bone marrow; MDA-MB-231, breast; Raji, lymphoblast; mesenchymal stem cell (MSC), bone marrow).

FIG. 31 shows a protein gel (left) and an anti-FLAG Western blot (right) of Chinese hamster ovary (CHO) cell-derived exosomes alone, or from cells overexpressing BASP1 or BASP1 N-terminal fragments (1-30 or 1-8) fused to FLAG-GFP.

### DETAILED DESCRIPTION

### Definitions

Unless defined otherwise, all technical and scientific terms used herein have the meaning commonly understood by a person skilled in the art to which this invention belongs. As used herein, the following terms have the meanings ascribed to them below.

As used herein, the term “extracellular vesicle” or “EV” refers to a cell-derived vesicle comprising a membrane that encloses an internal space. Extracellular vesicles comprise all membrane-bound vesicles that have a smaller diameter than the cell from which they are derived. Generally extracellular vesicles range in diameter from 20 nm to 1000 nm, and can comprise various macromolecular payload either within the internal space, displayed on the external surface of the extracellular vesicle, and/or spanning the membrane. Said payload can comprise nucleic acids, proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The exosome can be derived from a producer cell, and isolated from the producer cell based on its size, density, biochemical parameters, or a combination thereof.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

As used herein the term “lumen-engineered exosome” refers to an exosome with an internal luminal space modified in its composition. For example, the lumen is modified in its composition of a protein, a lipid, a small molecule, a carbohydrate, etc. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering.

As used herein the term “a modification” of a protein refers to a protein having at least 15% identity to the non-mutant amino acid sequence of the protein. A modification of a protein includes a fragment or a variant of the protein. A modification of a protein can further include chemical, or physical modification to a fragment or a variant of the protein.

As used herein the term “a fragment” of a protein refers to a protein that is N- and/or C-terminally deleted in comparison to the naturally occurring protein. Preferably, a fragment of MARCKS, MARCKSL1, or BASP1 retains the ability to be specifically targeted to the lumen of exosomes. Such a fragment is also referred to as a “functional fragment”. Whether a fragment is a functional fragment in that sense can be assessed by any art known methods to determine the protein content of exosomes including Western Blots, FACS analysis and fusions of the fragments with autofluorescent proteins like, e.g., GFP. In a particular embodiment the fragment of MARCKS, MARCKSL1, or BASP1 retains at least 50%, 60%, 70%, 80%, 90% or 100% of the ability of the naturally occurring MARCKS, MARCKSL1, or BASP1 to be specifically targeted to exosomes. In a particular embodiment the ability of the variant of MARCKS, MARCKSL1, BASP1 or of a fragment of MARCKS, MARCKSL1, or BASP1 is at least 70%, 80%, 85%, 90%, 95% or 99% of the ability of MARCKS, MARCKSL1, and BASP1, respectively, to be specifically targeted to the lumen of exosomes. This ability can be assessed, e.g. by fluorescently labeled variants, in the assays described in the experimental section.

As used herein the term “variant” of a protein refers to a protein that shares a certain amino acid sequence identity with another protein upon alignment by a method known in the art. A variant of a protein can include a substitution, insertion, deletion, frameshift or rearrangement in another protein. In a particular embodiment, the variant is a variant having at least 70% identity to MARCKS, MARCKSL1, BASP1 or a fragment of MARCKS, MARCKSL1, or BASP1. In some embodiments variants or variants of fragments of MARCKS share at least 70%, 80%, 85%, 90%, 95% or 99% sequence identity with MARCKS according to SEQ ID NO: 1 or with a functional fragment thereof. In some embodiments variants or variants of fragments of MARCKSL1 share at least 70%, 80%, 85%, 90%, 95% or 99% sequence identity with MARCKSL1 according to SEQ ID NO: 2 or with a functional fragment thereof. In some embodiments variants or variants of fragments of BASP1 share at least 70%, 80%, 85%, 90%, 95% or 99% sequence identity with BASP1 according to SEQ ID NO: 3 or with a functional fragment thereof. In each of above cases, it is preferred that the variant or variant of a fragment retains the ability to be specifically targeted to the lumen of exosomes.

Methods of alignment of sequences for comparison are well-known in the art. Various programs and alignment algorithms are described in: Smith and Waterman, Adv. Appl. Math. 2: 482 (1981); Needleman and Wunsch, J. Mol. Bio. 48: 443 (1970); Pearson and Lipman, Methods in Mol. Biol. 24: 307-31 (1988); Higgins and Sharp, Gene 73: 15 237-44 (1988); Higgins and Sharp, CABIOS 5: 151-3 (1989) Corpet et al., Nuc. Acids Res. 16: 10881-90 (1988); Huang et al., Comp. Appl. BioSci. 8: 155-65 (1992); and Pearson et al., Meth. Mol. Biol. 24: 307-31 (1994). The NCBI Basic Local Alignment Search Tool (BLAST) [Altschul 20 et al., J. Mol. Biol. 215: 403-10 (1990) J is available from several sources, including the National Center for Biological Information (NBCl, Bethesda, Md.) and on the Internet, for use in connection with the sequence analysis programs blastp, blasm, blastx, tblastn and tblastx. BLAST and a description of how to determine sequence identify using the program can be accessed at the official website of NCBI (National Center for Biotechnology Information) under NIH (National Institute of Health).

Recitation of any protein provided herein encompasses a functional variant of the protein. The term “functional variant” of a protein refers to a variant of the protein that retains the ability to be specifically targeted to the lumen of exosomes. In a particular embodiment the ability of the functional variant of MARCKS, MARCKSL1, BASP1 or of a fragment of MARCKS, MARCKSL1, or BASP1 is at least 70%, 80%, 85%, 90%, 95% or 99% of the ability of MARCKS, MARCKSL1, and BASP1, respectively, to be specifically targeted to the lumen of exosomes.

As used herein the term “producer cell” refers to a cell used for generating an exosome. A producer cell can be a cell cultured in vitro, or a cell in vivo. A producer cell includes, but not limited to, a cell known to be effective in generating exosomes, e.g., HEK293 cells, Chinese hamster ovary (CHO) cells, and mesenchymal stem cells (MSCs).

As used herein the term “target protein” or “target peptide” refers to a protein or peptide that can be targeted to the lumen of an exosome. The target protein or peptide can be a non-mutant protein that is naturally targeted to an exosome lumen, or a fragment or a modification of the non-mutant protein. The target protein can be a fusion protein containing a flag tag, a therapeutic peptide, a targeting moiety, or other peptide attached to the non-mutant protein or a modification or a fragment of the non-mutant protein. The target protein can comprise a modification such as myristoylation, prenylation, or palmitoylation, or a soluble protein attached to the internal leaflet of the membrane by a linker.

As used herein the term “cargo protein” or cargo peptide” refers to any protein or peptide, or fragment or modification thereof, which can be loaded into an exosome or engineered exosome. Cargo proteins or peptide may include therapeutic peptides or proteins that act on a target (e.g. a target cell) that is contacted with the exosome. Cargo proteins may be a fusion protein comprising a targeting protein or peptide or fragment or modification thereof, as described above, such that the cargo fusion protein can be targeted to an exosome lumen.

As used herein the term “contaminant protein” refers to a protein that is not associated with an exosome. For example, a contaminant protein includes a protein, not enclosed in the exosome and not attached to or incorporated into the membrane of the exosome.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired exosome preparation. In some embodiments, isolating or purifying as used herein is the process of removing, partially removing (e.g., a fraction) of the exosomes from a sample containing producer cells. In some embodiments, an isolated exosome composition has no detectable undesired activity or, alternatively, the level or amount of the undesired activity is at or below an acceptable level or amount. In other embodiments, an isolated exosome composition has an amount and/or concentration of desired exosomes at or above an acceptable amount and/or concentration. In other embodiments, the isolated exosome composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some embodiments, isolated exosome preparations are substantially free of residual biological products. In some embodiments, the isolated exosome preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the exosome composition contains no detectable producer cells and that only exosomes are detectable.

The term “excipient” or “carrier” refers to an inert substance added to a pharmaceutical composition to further facilitate administration of a compound. The term “pharmaceutically-acceptable carrier” or “pharmaceutically-acceptable excipient” encompasses any of the agents approved by a regulatory agency of the US Federal government or listed in the US Pharmacopeia for use in animals, including humans, as well as any carrier or diluent that does not cause significant irritation to a subject and does not abrogate the biological activity and properties of the administered compound. Included are excipients and carriers that are useful in preparing a pharmaceutical composition and are generally safe, non-toxic, and desirable.

As used herein, the term “payload” refers to a therapeutic agent that acts on a target (e.g., a target cell) that is contacted with the EV. Payloads that can be introduced into an exosome and/or a producer cell include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, viruses and viral particles (e.g., adeno-associated viruses and viral particles, retroviruses, adenoviruses, etc.) and small molecules (e.g., small molecule drugs and toxins, including small molecule STING agonists including cyclic dinucleotides such as ML-RR S2 and 3′-3′ cAIMPdFSH).

As used herein, “a mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

The terms “individual,” “subject,” “host,” and “patient,” are used interchangeably herein and refer to any mammalian subject for whom diagnosis, treatment, or therapy is desired, particularly humans. The methods described herein are applicable to both human therapy and veterinary applications. In some embodiments, the subject is a mammal, and in other embodiments the subject is a human.

As used herein, the term “substantially free” means that the sample comprising exosomes comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, the term “macromolecule” means nucleic acids, exogenous proteins, lipids, carbohydrates, metabolites, or a combination thereof.

As used herein, the term “conventional exosome protein” means a protein previously known to be enriched in exosomes, including but not limited to CD9, CD63, CD81, PDGFR, GPI anchor proteins, lactadherin LAMP2, and LAMP2B, a fragment thereof, or a peptide that binds thereto. For the avoidance of doubt, PTGFRN, BSG, IGSF2, IGSF3, IGSF8, ITGB1, ITGA4, SLC3A2, ATP transporter or a fragment or a variant thereof are not conventional exosome proteins.

Other Interpretational Conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

Unless otherwise indicated, reference to a compound that has one or more stereocenters intends each stereoisomer, and all combinations of stereoisomers, thereof.

Exosome Proteins

Some embodiments of the present invention relate to identification, use and modification of exosome proteins, which are highly enriched in exosome lumens. Such exosome proteins can be identified by analyzing highly purified exosomes with mass spectrometry or other methods known in the art.

The proteins include various luminal proteins or membrane proteins, such as transmembrane proteins, integral proteins and peripheral proteins, enriched on the exosome membranes. Specifically, the proteins include, but not limited to, (1) myristoylated alanine rich Protein Kinase C substrate (MARCKS); (2) myristoylated alanine rich Protein Kinase C substrate like 1 (MARCKSL1); and (3) brain acid soluble protein 1 (BASP1).

One or more exosome proteins identified herein can be selectively used depending on a producer cell, production condition, purification methods, or intended application of the exosomes. Exosome proteins enriched in the lumen of certain exosomes with a specific size range, a targeting moiety, a charge density, a payload, etc. can be identified and used in some embodiments of the present invention. In some embodiments, more than one exosome proteins can be used concurrently or subsequently for generation and isolation of therapeutic exosomes.

Lumen-Engineered Exosomes

Another aspect of the present invention relates to generation and use of lumen-engineered exosomes. Lumen-engineered exosomes have an internal space modified in its compositions. For example, the composition of the lumen can be modified by changing the protein, lipid or glycan content of the lumen.

In some embodiments, the lumen-engineered exosomes are generated by chemical and/or physical methods, such as PEG-induced fusion and/or ultrasonic fusion.

In other embodiments, the lumen-engineered exosomes are generated by genetic engineering. Exosomes produced from a genetically-modified producer cell or a progeny of the genetically-modified cell can contain modified lumen compositions. In some embodiments, lumen-engineered exosomes have the exosome protein at a higher or lower density or include a modification or a fragment of the exosome protein.

For example, lumen-engineered exosomes can be produced from a cell transformed with an exogenous sequence encoding the exosome protein or a modification or a fragment of the exosome protein. Exosomes including proteins expressed from the exogenous sequence can include modified lumen protein compositions.

Various modifications or fragments of the exosome protein can be used for the embodiments of the present invention. For example, proteins modified to be more effectively targeted to exosome lumens can be used. Proteins modified to comprise a minimal fragment required for specific and effective targeting to exosome lumens can be also used.

Fusion proteins having a therapeutic activity can be also used. For example, the fusion protein can comprise MARCKS, MARCKSL1, BASP1, or a modification thereof, in particular a fragment or variant thereof, and a therapeutic peptide or a cargo protein or peptide. In some embodiments, the fusion protein comprises a fragment of the amino terminus of BASP1.

The therapeutic peptide is selected from a group consisting of a natural peptide, a recombinant peptide, a synthetic peptide, or a linker to a therapeutic compound. The therapeutic compound can be nucleotides, amino acids, lipids, carbohydrates, or small molecules. The therapeutic peptide can be an antibody, an enzyme, a ligand, an antigen (e.g., a tumor antigen or an antigen from an infectious agent such as a bacteria, virus, fungus, or protozoa), a receptor, an antimicrobial peptide, a transcription factor, or a fragment or a modification thereof. The fusion proteins can be presented in the lumen of exosomes and provide a therapeutic activity to the exosome.

In some embodiments, the therapeutic peptide is a component of a genome editing complex. In some embodiments, said genome editing complex is a transcription activator-like effector nuclease (TAL-effector nuclease or TALEN); a zinc finger nuclease (ZFN); a recombinase; a CRISPR/Cas9 complex, a CRISPR/Cpfl complex, a CRISPR/C2c1, C2c2, or C2c3 complex, a CRISPR/CasY or CasX complex, or any other appropriate CRISPR complex known in the art; or any other appropriate genome editing complex known in the art or any combination thereof.

In some embodiments, the therapeutic peptide is a transmembrane peptide. The transmembrane peptides described herein may be expressed as fusion proteins to any of the sequences described herein or any fragments or variants thereof. In some embodiments, the transmembrane protein has a first end fused to the luminal sequence in the lumen of the exosome, and a second terminus expressed on the surface of the exosome. In some embodiments, the transmembrane protein comprises PTGFRN, BSG, IGSF2, IGSF3, IGSF8, ITGB1, ITGA4, SLC3A2, ATP transporter or a fragment or a variant thereof.

In some embodiments, the therapeutic peptide is a nucleic acid binding protein. In some embodiments, the nucleic acid binding protein is Dicer, an Argonaute protein, TRBP, MS2 bacteriophage coat protein. In some embodiments, the nucleic acid binding protein additionally comprises one or more RNA or DNA molecules. In some embodiments, the one or more RNA is a miRNA, siRNA, guide RNA, lincRNA, mRNA, antisense RNA, dsRNA, or combinations thereof.

In some embodiments, the therapeutic peptide is a part of a protein-protein interaction system. In some embodiments, the protein-protein interaction system comprises an FRB-FKBP interaction system, e.g., the FRB-FKBP interaction system as described in Banaszynski et al., J Am Chem Soc. 2005 Apr. 6; 127(13):4715-21.

The fusion proteins can be targeted to the lumen of exosomes and provide a therapeutic activity to the exosome.

In some embodiments, fusion proteins having a targeting moiety are used. For example, fusion proteins can comprise MARCKS, MARCKSL1, BASP1, or a fragment or a modification thereof, and a targeting moiety. The targeting moiety can be used for targeting the exosome to a specific organ, tissue, or cell for a treatment using the exosome. In some embodiments, the targeting moiety is an antibody or antigen-binding fragment thereof. Antibodies and antigen-binding fragments thereof include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, anti-idiotype antibodies, antibody fragments, such as, e.g., scFv, (scFv)₂, Fab, Fab′, and F(ab′)₂, F(ab1)₂, Fv, dAb, and Fd fragments, diabodies, and antibody-related polypeptides. Antibodies and antigen-binding fragments thereof also includes bispecific antibodies and multispecific antibodies so long as they exhibit the desired biological activity or function.

In some embodiments, fusion proteins comprising viral proteins are used. In some embodiments, the fusion protein comprises viral capsid or envelope proteins. In some embodiments, the fusion proteins allow for the assembly of intact viruses that are retained in the lumen of an exosome.

In some embodiments, fusion proteins that comprise MARCKS, MARCKSL1, BASP1, any of SEQ ID NO: 4-109 or a modification thereof, in particular a fragment or variant thereof, resulting in enrichment of the fusion protein in exosomes compared to expression of the fusion protein lacking MARCKS, MARCKSL1, BASP1, any of SEQ ID NO: 4-109 or a modification thereof, in particular a fragment or variant thereof, or compared to fusion proteins that comprise conventional exosome proteins. In some embodiments, the fusion proteins that comprise MARCKS, MARCKSL1, BASP1, any of SEQ ID NO: 4-109 or a fragment or a modification thereof comprise a peptide with the sequence MGXKLSKKK, where X is alanine or any other amino acid (SEQ ID NO: 117); or a peptide with sequence of (M)(G)(π)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In some embodiments, the fusion protein comprises a peptide with sequence of (M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, 1 is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In some embodiments, the conventional exosome protein is selected from the list consisting of CD9, CD63, CD81, PDGFR, GPI anchor proteins, LAMP2, LAMP2B, and a fragment thereof. In some embodiments, the enrichment of the fusion protein comprising MARCKS, MARCKSL1, BASP1, any of SEQ ID NO: 4-109 or a fragment or a modification thereof in exosomes is >2-fold, >4-fold, >8-fold, >16-fold, >25-fold, >50-fold, >100-fold, >200-fold, >500-fold, >750-fold, >1,000-fold, >2,000-fold, >5,000-fold, >7,500-fold, >10,000-fold higher than the fusion protein lacking MARCKS, MARCKSL1, BASP1, any of SEQ ID NO: 4-109 or a fragment or a modification thereof, or compared to fusion proteins that comprise conventional exosome proteins. In some embodiments, the protein sequence of any of SEQ ID NO: 1-109 is sufficient to load the exosomes with the fusion protein.

In some embodiments, the lumen-engineered exosome comprising a fusion protein containing an exogenous sequence and an exosome lumen protein newly-identified herein has a higher density of the fusion protein than similarly engineered exosomes comprising an exogenous sequence conjugated to a conventional exosome protein known in the art (e.g., CD9, CD63, CD81, PDGFR, GPI anchor proteins, lactadherin, LAMP2, and LAMP2B, a fragment thereof, or a peptide that binds thereto). In some embodiments, said fusion protein containing an exosome protein newly-identified herein is present at 2-, 4-, 8-, 16-, 32-, 64-, 100-, 200-, 400-, 800-, 1,000-fold or a higher density in the exosome lumen than fusion proteins in other exosome lumens similarly modified using a conventional exosome protein. In some embodiments, said fusion protein containing an exosome protein newly-identified herein is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density in the exosome lumen than fusion proteins in other exosome lumens similarly modified using a conventional exosome protein.

In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD9. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD63. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD81. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using PDGFR. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using GPI anchor proteins. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using lactadherin. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2B. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using an conventional protein. In some embodiments, a fusion protein comprising MARCKS, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using a variant of a conventional exosome protein.

In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD9. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD63. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD81. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using PDGFR. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using GPI anchor proteins. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using lactadherin. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2B. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using an conventional protein. In some embodiments, a fusion protein comprising MARCKSL1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using a variant of a conventional exosome protein.

In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD9. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD63. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD81. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using PDGFR. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using GPI anchor proteins. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using lactadherin. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2B. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using an conventional protein. In some embodiments, a fusion protein comprising BASP1, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using a variant of a conventional exosome protein.

In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD9. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD63. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using CD81. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using PDGFR. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using GPI anchor proteins. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using lactadherin. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using LAMP2B. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using an conventional protein. In some embodiments, a fusion protein comprising any of SEQ ID NO: 1-109, a variant, a fragment, a variant of a fragment, or a modification thereof is present at 2-, 4-, 8-, 16-, 32-, 64-, 100-, 200-, 400-, 800-, 1,000-fold or a higher density than the exosomes similarly modified using a variant of a conventional exosome protein.

In some embodiments, the lumen-engineered exosomes described herein demonstrate superior characteristics compared to lumen-engineered exosomes known in the art. For example, lumen-engineered exosomes produced by using the newly-identified exosome proteins provided herein contain modified proteins more highly enriched in their lumen than exosomes in prior art, e.g., those produced using conventional exosome proteins. Moreover, the lumen-engineered exosomes of the present invention can have greater, more specific, or more controlled biological activity compared to lumen-engineered exosomes known in the art. For example, a lumen-engineered exosome comprising a therapeutic or biologically relevant exogenous sequence fused to an exosome protein or a fragment thereof described herein (e.g., BASP1 or a fragment thereof) can have more of the desired engineered characteristics than fusion to scaffolds known in the art. Scaffold proteins known in the art include tetraspanin molecules (e.g., CD63, CD81, CD9 and others), lysosome-associated membrane protein 2 (LAMP2 and LAMP2B), platelet-derived growth factor receptor (PDGFR), GPI anchor proteins, lactadherin and fragments thereof, and peptides that have affinity to any of these proteins or fragments thereof. For the avoidance of doubt, PTGFRN, BSG, IGSF2, IGSF3, IGSF8, ITGB1, ITGA4, SLC3A2, ATP transporter or a fragment or a variant thereof are not conventional exosome proteins. Previously, overexpression of exogenous proteins relied on stochastic or random disposition of the exogenous proteins into the exosome for producing lumen-engineered exosomes. This resulted in low-level, unpredictable density of the exogenous proteins in exosomes. Thus, the exosome proteins and fragments thereof described herein provide important advancements in novel exosome compositions and methods of making the same.

Fusion proteins provided herein can comprise MARCKS, MARCKSL1, BASP1, or a fragment or a variant thereof, and an additional peptide. The additional peptide can be attached to either the N terminus or the C terminus of the exosome protein or a fragment or a variant thereof.

In some embodiments, fusion proteins provided herein comprise MARCKS, MARCKSL1, BASP1, or a fragment or a variant thereof, and two additional peptides. Both of the two additional peptides can be attached to either the N terminus or the C terminus of the exosome protein or a fragment or a variant thereof. In some embodiments, one of the two additional peptides is attached to the N terminus and the other of the two additional peptides is attached to the C terminus of the exosome protein or a fragment or a variant thereof.

In some embodiments, the compositions and methods of generating lumen-engineered extracellular vesicles described herein comprise nanovesicles.

Producer Cell for Production of Lumen-Engineered Exosomes

Exosomes of the present invention can be produced from a cell grown in vitro or a body fluid of a subject. When exosomes are produced from in vitro cell culture, various producer cells, e.g., HEK293 cells, can be used for the present invention. Additional cell types that can be used for the production of the lumen-engineered exosomes described herein include, without limitation, mesenchymal stem cells, T-cells, B-cells, dendritic cells, macrophages, and cancer cell lines.

The producer cell can be genetically modified to comprise one or more exogenous sequences to produce lumen-engineered exosomes. The genetically-modified producer cell can contain the exogenous sequence by transient or stable transformation. The exogenous sequence can be transformed as a plasmid. The exogenous sequences can be stably integrated into a genomic sequence of the producer cell, at a targeted site or in a random site. In some embodiments, a stable cell line is generated for production of lumen-engineered exosomes.

The exogenous sequences can be inserted into a genomic sequence of the producer cell, located within, upstream (5′-end) or downstream (3′-end) of an endogenous sequence encoding the exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present invention.

The exogenous sequences can comprise a sequence encoding the exosome protein or a modification or a fragment of the exosome protein. An extra copy of the sequence encoding the exosome protein can be introduced to produce a lumen-engineered exosome having a higher density of the exosome protein. An exogenous sequence encoding a modification or a fragment of the exosome protein can be introduced to produce a lumen-engineered exosome containing the modification or the fragment of the exosome protein. An exogenous sequence encoding an affinity tag can be introduced to produce a lumen-engineered exosome containing a fusion protein comprising the affinity tag attached to the exosome protein.

In some embodiments, a lumen-engineered exosome has a higher density of the exosome protein than native exosomes isolated from the same or similar producer cell types. In some embodiments, said exosome protein is present at 2-, 4-, 8-, 16-, 32-, 64-, 100-, 200-, 400-, 800-, 1,000-fold or to a higher density on said lumen-engineered exosome than said native exosome. In some embodiments, said exosome protein is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than said native exosome. In some embodiments, a fusion protein comprising the exosome protein is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than the unmodified exosome protein on said native exosome. In some embodiments, a fragment or a variant of the exosome protein is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than the unmodified exosome protein on said native exosome.

In particular embodiments, MARCKS, a fragment or a variant of MARCKS, or a modification thereof is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than the unmodified MARCKS on said native exosome. In some embodiments, MARCKSL1, a fragment or a variant of MARCKSL1, or a modification thereof is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than the unmodified MARCKSL1 on said native exosome. In some embodiments, BASP1, a fragment or a variant of BASP1, or a modification thereof is present at 2 to 4-fold, 4 to 8-fold, 8 to 16-fold, 16 to 32-fold, 32 to 64-fold, 64 to 100-fold, 100 to 200-fold, 200 to 400-fold, 400 to 800-fold, 800 to 1,000-fold or to a higher density on said lumen-engineered exosome than the unmodified BASP1 on said native exosome.

In some embodiments, the producer cell is further modified to comprise an additional exogenous sequence. For example, an additional exogenous sequence can be included to modulate endogenous gene expression, or produce an exosome including a certain polypeptide as a payload. In some embodiments, the producer cell is modified to comprise two exogenous sequences, one encoding the exosome protein or a modification or a fragment of the exosome protein, and the other encoding a payload.

More specifically, lumen-engineered exosomes can be produced from a cell transformed with a sequence encoding one or more exosome lumen proteins including, but not limited to, (1) myristoylated alanine rich Protein Kinase C substrate (MARCKS); (2) myristoylated alanine rich Protein Kinase C substrate like 1 (MARCKSL1); and (3) brain acid soluble protein 1 (BASP1). Any of the one or more exosome lumen proteins described herein can be expressed from a plasmid, an exogenous sequence inserted into the genome or other exogenous nucleic acid such as a synthetic messenger RNA (mRNA).

In some embodiments, the one or more exosome lumen protein is expressed in a cell transformed with an exogenous sequence encoding its full length, endogenous form. In some embodiments, such an exogenous sequence encodes MARCKS protein of SEQ ID NO: 1. In some embodiments, such an exogenous sequence encodes MARCKSL1 protein of SEQ ID NO: 2. In some embodiments, such an exogenous sequence encodes BASP1 protein of SEQ ID NO:

3.

Lumen-engineered exosomes can be produced from a cell transformed with a sequence encoding a fragment of one or more exosome lumen proteins including, but not limited to, (1) myristoylated alanine rich Protein Kinase C substrate (MARCKS); (2) myristoylated alanine rich Protein Kinase C substrate like 1 (MARCKSL1); and (3) brain acid soluble protein 1 (BASP1). In some embodiments, the sequence encodes a fragment of the exosome lumen protein lacking at least 5, 10, 50, 100, 200, or 300 amino acids from the N-terminus of the native protein. In some embodiments, the sequence encodes a fragment of the exosome lumen protein lacking at least 5, 10, 50, 100, 200, or 300 amino acids from the C-terminus of the native protein. In some embodiments, the sequence encodes a fragment of the exosome lumen protein lacking at least 5, 10, 50, 100, 200, or 300 amino acids from both the N-terminus and C-terminus of the native protein. In some embodiments, the sequence encodes a fragment of the exosome lumen protein lacking one or more functional or structural domains of the native protein. In some embodiments, the fusion protein comprises a peptide of SEQ ID NO: 4-109. In some embodiments, the fusion protein comprises the peptide of SEQ ID NO: 13. In some embodiments, the fusion protein comprises a peptide with the sequence MGXKLSKKK, where X is alanine or any other amino acid (SEQ ID NO: 117). In some embodiments, the fusion protein comprises a peptide with sequence of (M)(G)(π)(ξ)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In some embodiments, the fusion protein comprises a peptide with sequence of (M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu).

In some embodiments, lumen-engineered exosomes can be produced from a cell transformed with a sequence encoding an exosome protein or a fragment or a modification thereof fused to one or more heterologous proteins. In some embodiments, the one or more heterologous proteins are fused to the N-terminus of the exosome protein or a modification thereof, in particular a fragment or variant thereof. In some embodiments, the one or more heterologous proteins are fused to the C-terminus of the exosome protein or a modification thereof, in particular a fragment or variant thereof. In some embodiments, the one or more heterologous proteins are fused to the N-terminus and the C-terminus of the exosome protein or a modification thereof, in particular a fragment or variant thereof. In some embodiments, the one or more heterologous proteins are mammalian proteins. In some embodiments, the one or more heterologous proteins are human proteins.

In some embodiments lumen-engineered exosomes are produced from a cell transformed with a sequence encoding a polypeptide of a sequence identical or similar to a full-length or a fragment of a native exosome lumen protein including, but not limited to, (1) myristoylated alanine rich Protein Kinase C substrate (MARCKS); (2) myristoylated alanine rich Protein Kinase C substrate like 1 (MARCKSL1); and (3) brain acid soluble protein 1 (BASP1). In some embodiments, said polypeptide is 50% identical to a full-length or a fragment of a native exosome lumen protein, e.g., 50% identical to SEQ ID NO: 1-3. In some embodiments, said polypeptide is 60% identical to a full-length or a fragment of a native exosome lumen protein, e.g., 60% identical to SEQ ID NO: 1-3. In some embodiments, said polypeptide is 70% identical to a full-length or a fragment of a native exosome lumen protein, e.g., 70% identical to SEQ ID NO: 1-3. In some embodiments, said polypeptide is 80% identical to a full-length or a fragment of a native exosome lumen protein, e.g., 80% identical to SEQ ID NO: 1-3. In some embodiments, said polypeptide is 90% identical to a full-length or a fragment of a native exosome lumen protein, e.g., 90% identical to SEQ ID NO: 1-3. In some embodiments, said polypeptide is 95% identical to a full-length or a fragment of a native exosome lumen protein, e.g., 95% identical to SEQ ID NO: 1-3. In some embodiments, said polypeptide is 99% identical to a full-length or a fragment of a native exosome lumen protein, e.g., 99% identical to SEQ ID NO: 1-3. In some embodiments, said polypeptide is 99.9% identical to a full-length or a fragment of a native exosome lumen protein, e.g., 99.9% identical to SEQ ID NO: 1-3.

In some embodiments, lumen-engineered exosomes produced from the cell comprise a polypeptide of a sequence identical or similar to a fragment of brain acid soluble protein 1 (BASP1). In some embodiments, said polypeptide is 50% identical to a full-length or a fragment of BASP1, e.g., 50% identical to SEQ ID NO: 4-109. In some embodiments, said polypeptide is 60% identical to a full-length or a fragment of BASP1, e.g., 60% identical to SEQ ID NO: 4-109. In some embodiments, said polypeptide is 70% identical to a full-length or a fragment of BASP1, e.g., 70% identical to SEQ ID NO: 4-109. In some embodiments, said polypeptide is 80% identical to a full-length or a fragment of BASP1, e.g., 80% identical to SEQ ID NO: 4-109. In some embodiments, said polypeptide is 90% identical to a full-length or a fragment of BASP1, e.g., 90% identical to SEQ ID NO: 4-109. In some embodiments, said polypeptide is 95% identical to a full-length or a fragment of BASP1, e.g., 95% identical to SEQ ID NO: 4-109. In some embodiments, said polypeptide is 99% identical to a full-length or a fragment of BASP1, e.g., 99% identical to SEQ ID NO: 4-109. In some embodiments, said polypeptide is 99.9% identical to a full-length or a fragment of BASP1, e.g., 99.9% identical to SEQ ID NO: 4-109. In some embodiments, said polypeptide is 100% identical to a fragment of BASP1, e.g., 100% identical to SEQ ID NO: 4-109.

Characterization of Exosomes

In some embodiments, the methods described herein further comprise the step of characterizing exosomes contained in each collected fraction. In some embodiments, contents of the exosomes can be extracted for study and characterization. In some embodiments, exosomes are isolated and characterized by metrics including, but not limited to, size, shape, morphology, or molecular compositions such as nucleic acids, proteins, metabolites, and lipids

### Measurement of the Contents of Exosomes

Exosomes can include proteins, peptides, RNA, DNA, and lipids. Total RNA can be extracted using acid-phenol:chloroform extraction. RNA can then be purified using a glass-fiber filter under conditions that recover small-RNA containing total RNA, or that separate small RNA species less than 200 nucleotides in length from longer RNA species such as mRNA. Because the RNA is eluted in a small volume, no alcohol precipitation step may be required for isolation of the RNA.

Exome compositions may be assessed by methods known in the art including, but not limited to, transcriptomics, sequencing, proteomics, mass spectrometry, or HP-LC.

The composition of nucleotides associated with isolated exosomes (including RNAs and DNAs) can be measured using a variety of techniques that are well known to those of skill in the art (e.g., quantitative or semi-quantitative RT-PCR, Northern blot analysis, solution hybridization detection). In a particular embodiment, the level of at least one RNA is measured by reverse transcribing RNA from the exosome composition to provide a set of target oligodeoxynucleotides, hybridizing the target oligodeoxynucleotides to one or more RNA-specific probe oligonucleotides (e.g., a microarray that comprises RNA-specific probe oligonucleotides) to provide a hybridization profile for the exosome composition, and comparing the exosome composition hybridization profile to a hybridization profile generated from a control sample. An alteration in the signal of at least one RNA in the test sample relative to the control sample is indicative of the RNA composition.

Also, a microarray can be prepared from gene-specific oligonucleotide probes generated from known RNA sequences. The array can contain two different oligonucleotide probes for each RNA, one containing the active, mature sequence and the other being specific for the precursor of the RNA (for example miRNA and pre-miRNAs). The array can also contain controls, such as one or more mouse sequences differing from human orthologs by only a few bases, which can serve as controls for hybridization stringency conditions. tRNAs and other RNAs (e.g., rRNAs, mRNAs) from both species can also be printed on the microchip, providing an internal, relatively stable, positive control for specific hybridization. One or more appropriate controls for non-specific hybridization can also be included on the microchip. For this purpose, sequences are selected based upon the absence of any homology with any known RNAs.

The microarray can be fabricated using techniques known in the art. For example, probe oligonucleotides of an appropriate length, e.g., 40 nucleotides, are 5′-amine modified at position C6 and printed on activated slides using commercially available microarray systems, e.g., the GeneMachine OmniGrid.™ 100 Microarrayer and Amersham CodeLink.™ Labeled cDNA oligomer corresponding to the target RNAs is prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g., 6.times. SSPE/30% formamide at 25° C. for 18 hours, followed by washing in 0.75.times. TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding complementary RNAs, in the exosome preparation. According to one embodiment, the labeled cDNA oligomer is a biotin-labeled cDNA, prepared from a biotin-labeled primer. The microarray is then processed by direct detection of the biotin containing transcripts using, e.g., Streptavidin-Alexa647 conjugate, and scanned utilizing conventional scanning methods. Image intensities of each spot on the array are proportional to the abundance of the corresponding RNA in the exosome.

Data mining work is completed by bioinformatics, including scanning chips, signal acquisition, image processing, normalization, statistic treatment and data comparison as well as pathway analysis. As such, microarray can profile hundreds and thousands of polynucleotides simultaneously with high throughput performance. Microarray profiling analysis of mRNA expression has successfully provided valuable data for gene expression studies in basic research. And the technique has been further put into practice in the pharmaceutical industry and in clinical diagnosis. With increasing amounts of miRNA data becoming available, and with accumulating evidence of the importance of miRNA in gene regulation, microarray becomes a useful technique for high through-put miRNA studies. The analysis of miRNA levels utilizing polynucleotide probes can be carried out in a variety of physical formats as well. For example, the use of microtiter plates or automation can be used to facilitate the processing of large numbers of test samples.

### Measurement of the Size of Exosomes

In some embodiments, the methods described herein comprise measuring the size of exosomes and/or populations of exosomes included in the purified fractions. In some embodiments, exosome size is measured as the longest measurable dimension. Generally, the longest general dimension of an exosome is also referred to as its diameter.

Exosome size can be measured using various methods known in the art, for example, nanoparticle tracking analysis, multi-angle light scattering, single angle light scattering, size exclusion chromatography, analytical ultracentrifugation, field flow fractionation, laser diffraction, tunable resistive pulse sensing, or dynamic light scattering.

Exosome size can be measured using dynamic light scattering (DLS) and/or multiangle light scattering (MALS). Methods of using DLS and/or MALS to measure the size of exosomes are known to those of skill in the art, and include the nanoparticle tracking assay (NTA, e.g., using a Malvern Nanosight NS300 nanoparticle tracking device). In a specific embodiment, the exosome size is determined using a Malvern NanoSight NS300. In some embodiments, the exosomes described herein have a longest dimension of about 20-1000 nm as measured by NTA (e.g., using a Malvern NanosightNS300). In other embodiments, the exosomes described herein have a longest dimension of about 40-1000 nm as measured by NTA (e.g., using a Malvern NanosightNS300). In other embodiments, the exosome populations described herein comprise a population, wherein 90% of said exosomes have a longest dimension of about 20-1000 nm as measured by NTA (e.g., using a Malvern Nanosight NS300). In other embodiments, the exosome populations described herein comprise a population, wherein 95% of said exosomes have a longest dimension of about 20-1000 nm as measured by NTA (e.g., using a Malvern Nanosight NS300). In other embodiments, the exosome populations described herein comprise a population, wherein 99% of said exosomes have a longest dimension of about 20-1000 nm as measured by NTA (e.g., using a Malvern Nanosight NS300). In other embodiments, the exosome populations described herein comprise a population, wherein 90% of said exosomes have a longest dimension of about 40-1000 nm as measured by NTA (e.g., using a Malvern Nanosight NS300). In other embodiments, the exosome populations described herein comprise a population, wherein 95% of said exosomes have a longest dimension of about 40-1000 nm as measured by NTA (e.g., using a Malvern Nanosight NS300). In other embodiments, the exosome populations described herein comprise a population, wherein 99% of said exosomes have a longest dimension of about 40-1000 nm as measured by NTA (e.g., using a Malvern Nanosight NS300).

Exosome size can be measured using tunable resistive pulse sensing (TRPS). In a specific embodiment, exosome size as measured by TRPS is determined using an iZON qNANO Gold. In some embodiments, the exosomes described herein have a longest dimension of about 20-1000 nm as measured by TRPS (e.g., using an iZON qNano Gold). In other embodiments, the exosomes described herein have a longest dimension of about 40-1000 nm as measured by TRPS (e.g., an iZON qNano Gold). In other embodiments, the exosome populations described herein comprise a population, wherein 90% of said exosomes have a longest dimension of about 20-1000 nm as measured by TRPS (e.g., using an iZON qNano Gold). In other embodiments, the exosome populations described herein comprise a population, wherein 95% of said exosomes have a longest dimension of about 20-1000 nm as measured by TRPS (e.g., using an iZON qNano Gold). In other embodiments, the exosome populations described herein comprise a population, wherein 99% of said exosomes have a longest dimension of about 20-1000 nm as measured by TRPS (e.g., using an iZON qNano Gold). In other embodiments, the exosome populations described herein comprise a population, wherein 90% of said exosomes have a longest dimension of about 40-1000 nm as measured by TRPS (e.g., using an iZON qNano Gold). In other embodiments, the exosome populations described herein comprise a population, wherein 95% of said exosomes have a longest dimension of about 40-1000 nm as measured by TRPS (e.g., using an iZON qNano Gold). In other embodiments, the exosome populations described herein comprise a population, wherein 99% of said exosomes have a longest dimension of about 40-1000 nm as measured by TRPS (e.g., using an iZON qNano Gold).

Exosome size can be measured using electron microscopy. In some embodiments, the method of electron microscopy used to measure exosome size is transmission electron microscopy. In a specific embodiment, the transmission electron microscope used to measure exosome size is a Tecnai™ G2 Spirit BioTWIN. Methods of measuring exosome size using an electron microscope are well-known to those of skill in the art, and any such method can be appropriate for measuring exosome size. In some embodiments, the exosomes described herein have a longest dimension of about 20-1000 nm as measured by a scanning electron microscope (e.g., a Tecnai™ G2 Spirit BioTWIN scanning electron microscope). In other embodiments, the exosomes described herein have a longest dimension of about 40-1000 nm as measured by a scanning electron microscope (e.g., a Tecnai™ G2 Spirit BioTWIN scanning electron microscope). In other embodiments, the exosome populations described herein comprise a population, wherein 90% of said exosomes have a longest dimension of about 20-1000 nm as measured by a scanning electron microscope (e.g., a Tecnai™ G2 Spirit BioTWIN scanning electron microscope). In other embodiments, the exosome populations described herein comprise a population, wherein 95% of said exosomes have a longest dimension of about 20-1000 nm as measured by a scanning electron microscope (e.g., a Tecnai™ G2 Spirit BioTWIN scanning electron microscope). In other embodiments, the exosome populations described herein comprise a population, wherein 99% of said exosomes have a longest dimension of about 20-1000 nm as measured by a scanning electron microscope (e.g., a Tecnai™ G2 Spirit BioTWIN scanning electron microscope). In other embodiments, the exosome populations described herein comprise a population wherein 90% of said exosomes have a longest dimension of about 40-1000 nm as measured by a scanning electron microscope (e.g., a Tecnai™ G2 Spirit BioTWIN scanning electron microscope). In other embodiments, the exosome populations described herein comprise a population wherein 95% of said exosomes have a longest dimension of about 40-1000 nm as measured by a scanning electron microscope (e.g., a Tecnai™ G2 Spirit BioTWIN scanning electron microscope). In other embodiments, the exosome populations described herein comprise a population wherein 99% of said exosomes have a longest dimension of about 40-1000 nm as measured by a scanning electron microscope (e.g., a Tecnai™ G2 Spirit BioTWIN scanning electron microscope).

Individual exosome size can be determined on a particle-by-particle basis by nano-flow cytometry. In some embodiments, the nano-flow cytometer is the Flow NanoAnalyzer (NanoFCM, Inc.; Xiamen, China). In some embodiments, the exosomes described herein have a longest dimension of about 20-1000 nm as measured by nano-flow cytometry (e.g., using a Flow NanoAnalyzer). In some embodiments, the exosomes described herein have a longest dimension of about 40-1000 nm as measured by nano-flow cytometry (e.g., using a Flow NanoAnalyzer). In some embodiments, the exosome populations described herein comprise a population, wherein 90% of said exosomes have a longest dimension of about 20-1000 nm as measured by nano-flow cytometry (e.g., using a Flow NanoAnalyzer). In some embodiments, the exosome populations described herein comprise a population, wherein 95% of said exosomes have a longest dimension of about 20-1000 nm as measured by nano-flow cytometry (e.g., using a Flow NanoAnalyzer). In some embodiments, the exosome populations described herein comprise a population, wherein 99% of said exosomes have a longest dimension of about 20-1000 nm as measured by nano-flow cytometry (e.g., using a Flow NanoAnalyzer). In some embodiments, the exosome populations described herein comprise a population, wherein 90% of said exosomes have a longest dimension of about 40-1000 nm as measured by nano-flow cytometry (e.g., using a Flow NanoAnalyzer). In some embodiments, the exosome populations described herein comprise a population, wherein 95% of said exosomes have a longest dimension of about 40-1000 nm as measured by nano-flow cytometry (e.g., using a Flow NanoAnalyzer). In some embodiments, the exosome populations described herein comprise a population, wherein 99% of said exosomes have a longest dimension of about 40-1000 nm as measured by nano-flow cytometry (e.g., using a Flow NanoAnalyzer).

### Measurement of the Charge Density of Exosomes

In some embodiments, the methods described herein comprise measuring the charge density of exosomes and/or populations of exosomes included in the purified fractions. In some embodiments, the charge density is measured by potentiometric titration, anion exchange, cation exchange, isoelectric focusing, zeta potential, capillary electrophoresis, capillary zone electrophoresis, or gel electrophoresis.

### Measurement of Density of Exosome Proteins

In some embodiments, the methods described herein comprise measuring the density of exosome proteins on the exosome surface. The surface density can be calculated or presented as the mass per unit area, the number of proteins per area, number of molecules or intensity of molecule signal per exosome, molar amount of the protein, etc. The surface density can be experimentally measured by methods known in the art, for example, by using bio-layer interferometry (BLI), FACS, Western blotting, fluorescence (e.g., GFP-fusion protein) detection, nano-flow cytometry, ELISA, alphaLISA, and/or densitometry by measuring bands on a protein gel.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations can be used, e.g., bp, base pair(s); kb, kilobase(s); pl, picoliter(s); s or sec, second(s); min, minute(s); h or hr, hour(s); aa, amino acid(s); nt, nucleotide(s); and the like.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 21th Edition (Easton, Pa.: Mack Publishing Company, 2005); Carey and Sundberg Advanced Organic Chemistry 3rd Ed. (Plenum Press) Vols A and B(1992).

### Example 1: Identification of Novel Exosome Proteins Collection of Exosomes

Exosomes were collected from the supernatant of high density suspension cultures of HEK293 SF cells after 9 days. The supernatant was filtered and fractionated by anion exchange chromatography and eluted in a step gradient of sodium chloride. The peak fraction with the highest protein concentration contained exosomes and contaminating cellular components. The peak fraction was isolated and further fractionated on an Optiprep™ density gradient by ultracentrifugation.

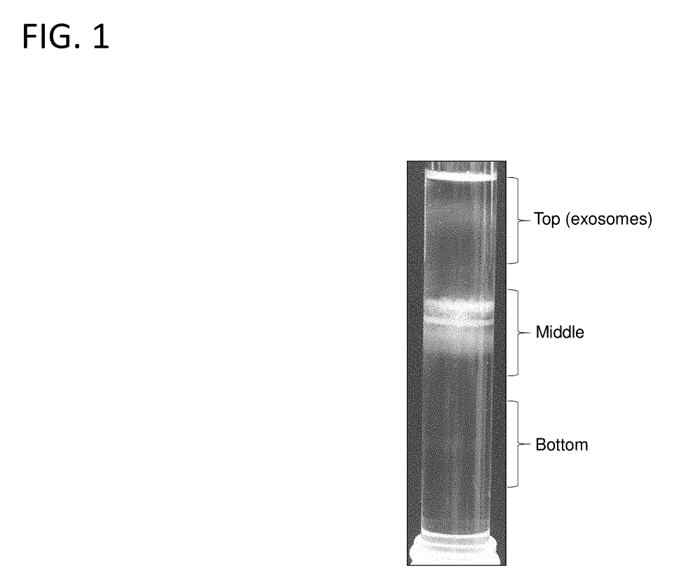

For the Optiprep™ gradient, a 4-tier sterile gradient was prepared with 4 mL 45% Optiprep™, 3 mL 30% Optiprep™, 2 mL 22.5% Optiprep™, 2 mL 17.5% Optiprep™, and 1 mL PBS in a 12 mL Ultra-Clear (344059) tube for a SW 41 Ti rotor. The exosome fraction was added to the Optiprep™ gradient and ultracentrifuged at 200,000×g for 16 hours at 4° C. to separate the exosome fraction. Ultracentrifugation resulted in a Top Fraction known to contain exosomes, a Middle Fraction containing cell debris of moderate density, and a Bottom Fraction containing high density aggregates and cellular debris (FIG. 1). The exosome layer was then gently collected from the top ˜3 mL of the tube.

The exosome fraction was diluted in ˜32 mL PBS in a 38.5 mL Ultra-Clear (344058) tube and ultracentrifuged at 133,900×g for 3 hours at 4° C. to pellet the purified exosomes. The pelleted exosomes were then resuspended in a minimal volume of PBS (˜200 μL) and stored at 4° C.

### Sample Preparation for LC-MS/MS Analysis

To determine proteins specific to exosomes, the Top Fraction and Bottom Fraction of the Optiprep™ gradient were analyzed by liquid chromatography-tandem mass spectrometry. Prior to analysis, the total protein concentration of the two samples was determined by bicinchoninic acid (BCA) assay, after which each sample was appropriately diluted to 125 μg/mL in PBS buffer. Next, 50.0 μL of each sample was added to a separate 1.5 mL microcentrifuge tube containing an equal volume of exosome lysis buffer (60 mM Tris, 400 mM GdmCl, 100 mM EDTA, 20 mM TCEP, 1.0% Triton X-100) followed by the transfer of 2.0 μL 1.0% Triton X-100 solution. All samples were then incubated at 55° C. for 60 minutes.

Protein precipitation was performed by adding 1250 μL of ethanol at −20° C. To improve efficiency, samples were vigorously vortexed and then sonicated in a water bath for 5 minutes. Precipitated material was pelleted by centrifuging for 5 minutes at 15,000 g at room temperature. The supernatant was decanted, and the pelleted material was thoroughly dried using nitrogen gas. Pellets were resuspended in 30.0 μL digestion buffer (30 mM Tris, 1.0 M GdmCl, 100 mM EDTA, 50 mM TCEP, pH 8.5) which also reduced disulfide bonds. Free cysteine residues were alkylated by adding 5.0 μL alkylation solution (375 mM iodoacetamide, 50 mM Tris, pH 8.5) and incubating the resulting solution at room temperature in the dark for at least 30 minutes.

After incubation, each sample was diluted using 30.0 μL 50 mM Tris pH 8.5, and proteolytic digestion was initiated by adding 2.0 μg trypsin. All samples were mixed and then incubated overnight at 37° C. After the incubation, trypsin activity was ceased by adding 5.0 μL 10% formic acid. Prior to analysis by LC-MS/MS, each sample was desalted using Pierce C18 spin columns. At the end of this process, each sample was dried down and reconstituted in 75.0 μL of 95:5 water:acetonitrile with 0.1% formic acid and transferred to an HPLC vial for analysis.

LC-MS/MS Analysis

Samples were injected into an UltiMate 3000 RSCLnano (Thermo Fisher Scientific) low flow chromatography system, and tryptic peptides were loaded onto an Acclaim PepMap 100 C18 trapping column (75 μm×2 cm, 3 μm particle size, 100 Å pore size, Thermo Fisher Scientific) using loading mobile phase (MPL: 95% water, 5% acetonitrile, 0.1% formic acid) at a flowrate of 2.500 μL/min. Peptides were eluted and separated with a gradient of mobile phase A (MPA: water, 0.1% formic acid) and mobile phase B (MPB: acetonitrile, 0.1% formic acid) at a flowrate of 300 nL/min across an EASY-Spray LC C18 analytical column (75 μm×25 cm, 2 μm particle size, 100 Å pore size, Thermo Fisher Scientific). The stepwise gradient used for elution began at 5% MPB, where it was held for 15 minutes during loading. The percentage MPB then increased from 5-17% over 30 minutes, again from 17-25% over 45 minutes, and finally from 25-40% over 5 minutes. The most hydrophobic species were removed by increasing to 90% MPB over 5 minutes, then holding there for 9 minutes. The total runtime for the method was 130 minutes and allowed a sufficient amount of time for column re-equilibration. Wash cycles were performed in between analytical injections to minimize carry-over.

Mass analyses were performed with a Q Exactive Basic (Thermo Fisher Scientific) mass spectrometer. Precursor ion mass spectra were measured across an m/z range of 400-1600 Da at a resolution of 70,000. The 10 most intense precursor ions were selected and fragmented in the HCD cell using a collision energy of 27, and MS/MS spectra were measured across an m/z range of 200-2000 Da at a resolution of 35,000. Ions with charge states from 2-4 were selected for fragmentation and the dynamic exclusion time was set to 30 seconds. An exclusion list containing 14 common polysiloxanes was utilized to minimize misidentification of known contaminants.

Data Processing

Proteins were first identified and quantified (label-free) using Proteome Discoverer software (version 2.1.1.21, Thermo Fisher Scientific) and the Sequest HT algorithm combined with the Target Decoy PSM Validator. Searches were performed against either the full Uniprot Homo sapiens (taxonomy 9606: 127,783 entries) or Swiss-Prot Homo sapiens (taxonomy 9606 version 2017-05-10: 42,153 entries) reference database, as well as a custom Uniprot database containing E1a proteins (7 entries). The following search parameters were used: enzyme, trypsin; maximum of 2 missed cleavages; minimum peptide length of 6 residues; 10 ppm precursor mass tolerance; and 0.02 Da fragment mass tolerance. The search also included specific dynamic modifications (oxidation of M; deamidation of N or Q; phosphorylation of S, T, or Y; pyro-glutamation of peptide-terminal E; and acetylation of protein N terminus) and static modifications (carbamidomethylation of C).

In the Target Decoy PSM Validator, the maximum delta Cn and both strict and relaxed target false discovery rates (FDRs) were set to 1 because the data were searched again using Scaffold software (version 4.8.2, Proteome Software Inc.). In Scaffold, the data were also searched using the X! Tandem open source algorithm to identify proteins using a protein threshold of 99.0%, a minimum of 2 peptides, and a peptide threshold of 95%.

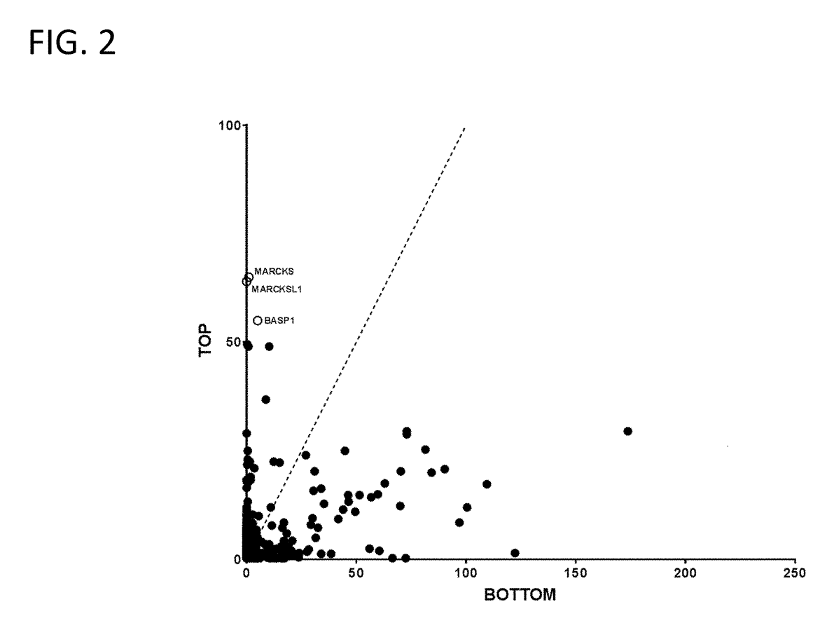

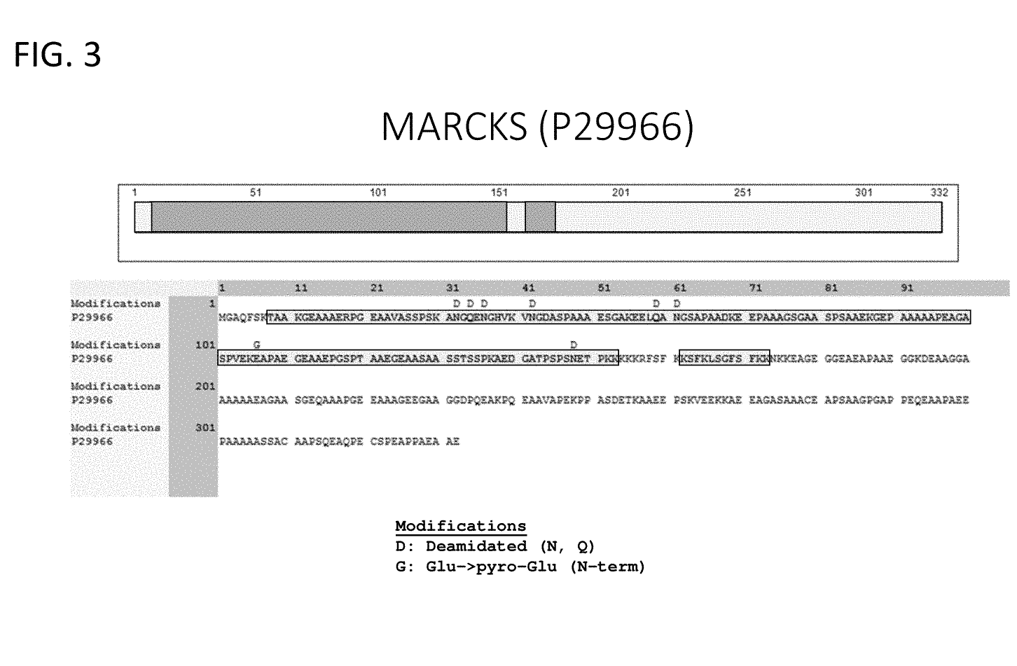

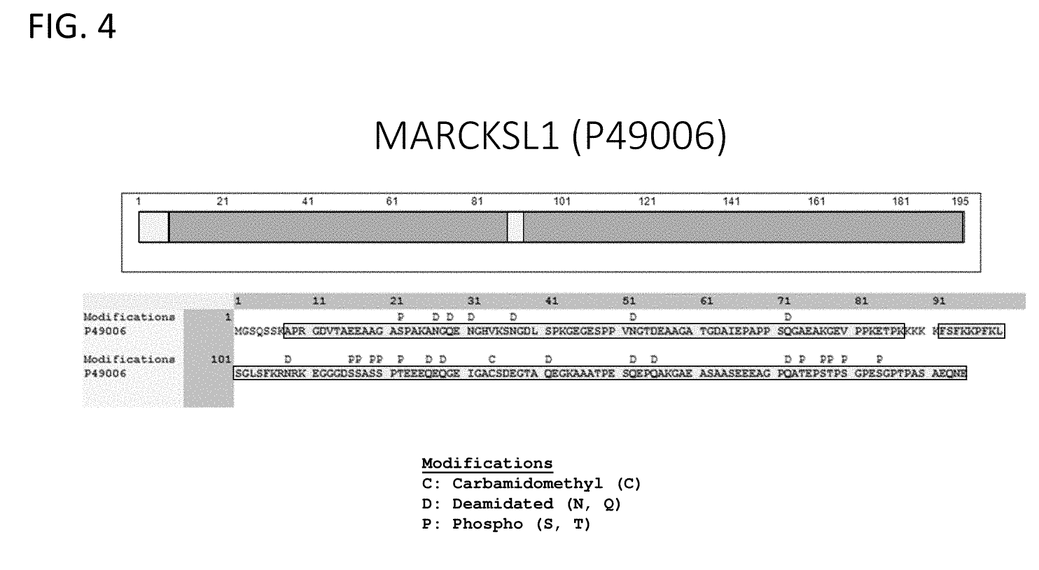

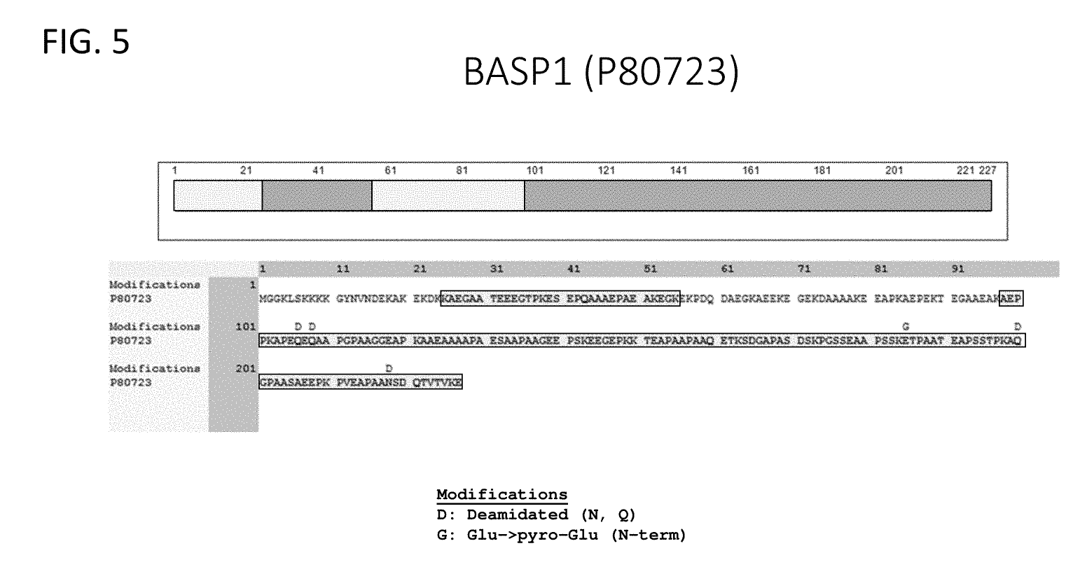

To determine the identity of novel exosome-specific proteins, total peptide spectral matches (PSMs) were compared for proteins found in the top exosome fraction of the Optiprep™ gradient versus those in the lower fraction. As shown in FIG. 2, there was weak correlation between the top-fraction proteins (Y-axis) and the bottom-fraction proteins (X-axis). Proteins plotted above the dotted line represent exosome-enriched proteins, while those below the dotted line represent contaminant-enriched proteins. Importantly, there were a number of proteins identified that lacked transmembrane domains, and which were highly enriched in the exosomes fraction, including (1) myristoylated alanine rich Protein Kinase C substrate (MARCKS); (2) myristoylated alanine rich Protein Kinase C substrate like 1 (MARCKSL1); and (3) brain acid soluble protein 1 (BASP1). As shown in the tryptic peptide coverage maps in FIG. 3-5, the mass spectrometry study resulted in broad coverage of MARCKS (FIG. 3), MARCKSL1 (FIG. 4), and BASP1 (FIG. 5). Since none of these proteins are predicted to have transmembrane domains, it suggests that they are enriched as soluble proteins in the lumen of exosomes. Together, these results demonstrate that there are numerous luminal proteins enriched in purified exosome populations that may be useful as payload scaffolds in generating engineered exosomes.

### Example 2: Verification of Lumen Protein Expression

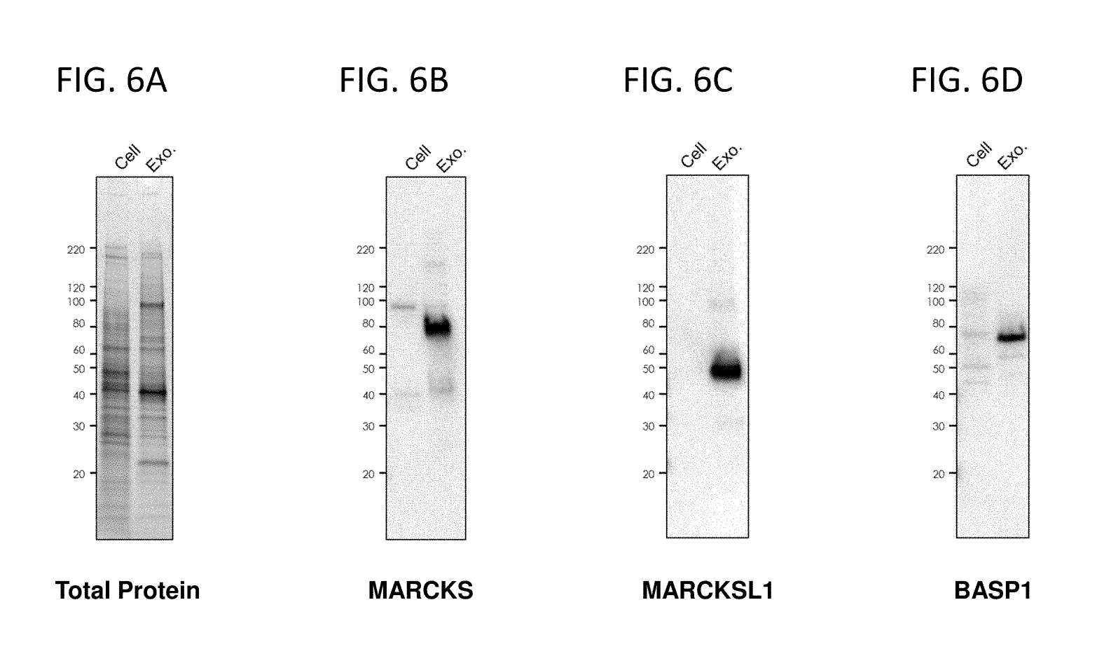

To confirm that the exosome-specific proteins identified in the mass spectrometry studies were highly expressed in the lumen of exosomes, Western blotting was carried out on total cell lysate and purified exosome populations from HEK293 cells. As shown in FIG. 6A, equal amounts of total protein from cell lysate (left) and purified exosomes (right) were loaded on a denaturing polyacrylamide gel. Western blotting for MARCKS (FIG. 6B), MARCKSL1 (FIG. 6C), and BASP1 (FIG. 6D) demonstrated that the bands representing the novel luminal proteins were easily detected in exosomes but not cell lysates, demonstrating that these proteins are highly enriched in exosomes, and may be visually detectable in total exosome lysate. The demonstration that these lumen proteins are highly expressed and enriched in exosomes provides an opportunity for generating lumen-modified exosomes containing heterologous proteins fused to any of these novel proteins at high levels.

### Example 3: Verification of Luminal Loading Using Novel Proteins as

Scaffolds

To confirm the utility of MARCKS, MARCKSL1, and/or BASP1 as luminal loading scaffolds, each of the proteins was fused to the N-terminus of GFP. Additionally, the first 30 amino acids of each of these proteins were also fused to GFP to determine whether a shorter protein fragment could drive loading of engineered exosomes. Exosomes engineered to contain CD81 (a well-established exosome marker) or PDGFR (a transmembrane protein with moderate exosome loading efficiency) fused to GFP were used as reference standards.

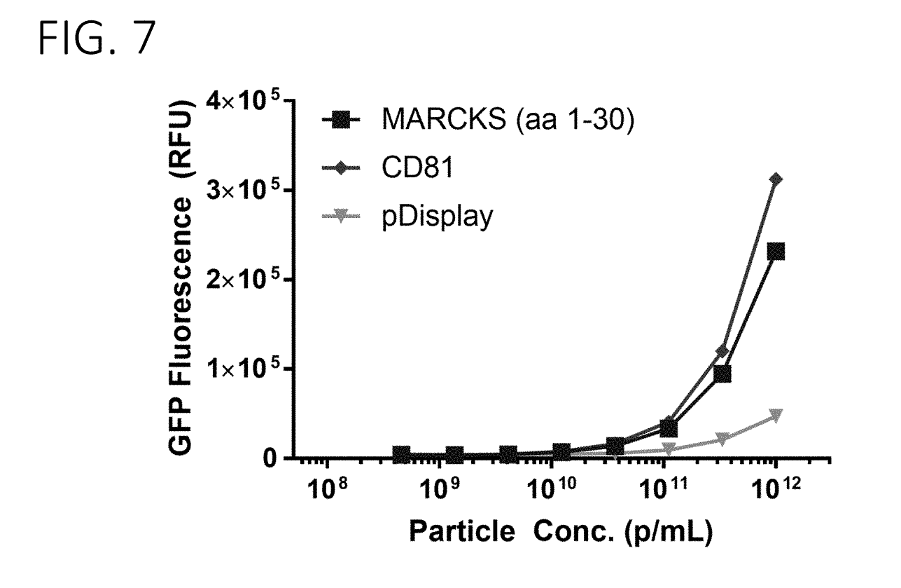

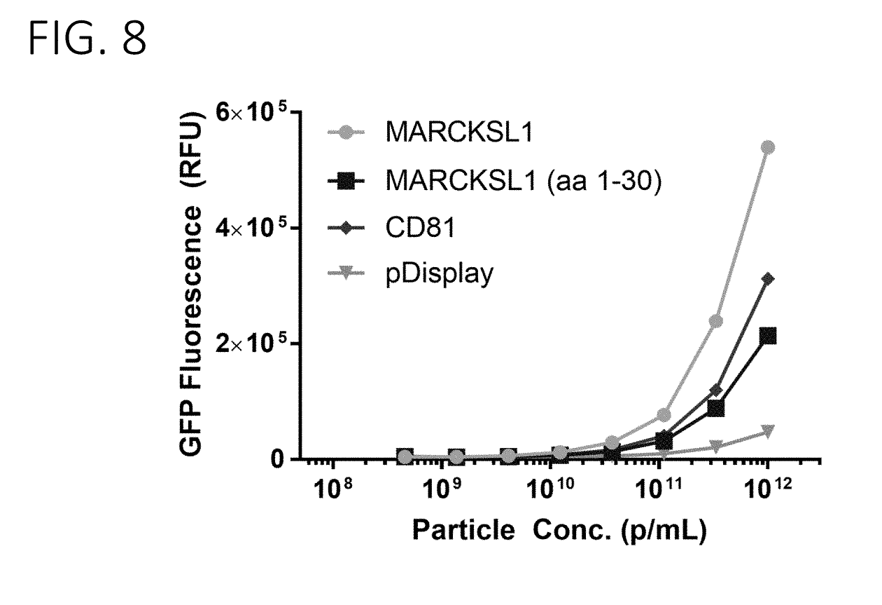

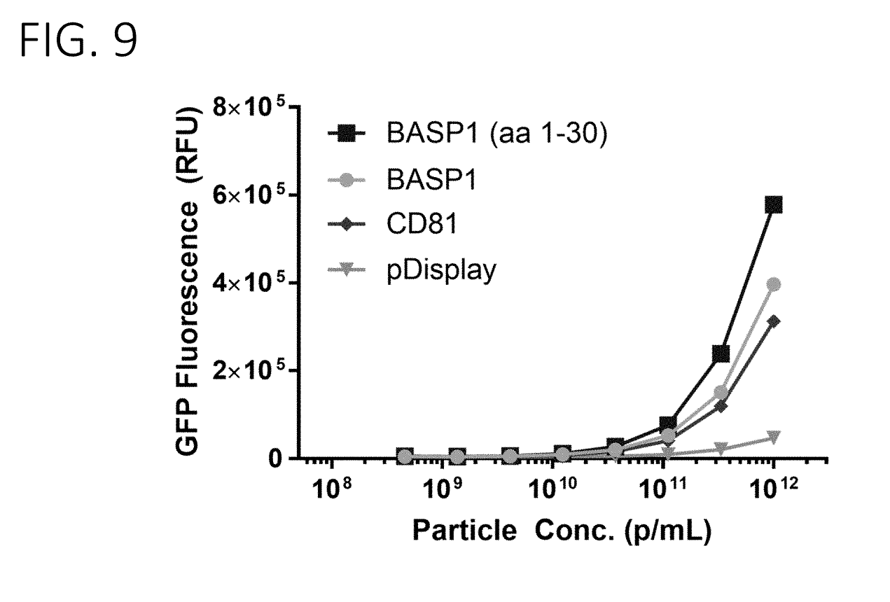

Engineered HEK293SF cells containing each of the expression constructs were stably selected and grown to high density in 200 ml cultures. The supernatants were collected and purified by Optiprep™ density gradient ultracentrifugation as described in Example 1. The resulting GFP-containing exosomes were measured in 96-well format on a Synergy H1 plate reader (BioTek®). As shown in FIG. 7, the first 30 amino acids of MARCKS fused to GFP (“MARCKS (aa 1-30)”) was insufficient to load exosomes above the level of either CD81-GFP (“CD81”) or PDGFR-GFP (“pDisplay”). Similarly, the first 30 amino acids of MARCKSL1 fused to GFP (“MARCKSL1 (aa 1-30)”) was insufficient to increase exosome loading compared to CD81-GFP (“CD81”), although the full length MARCKSL1-GFP fusion (“MARCKSL1”) led to dramatically higher signal than CD81-GFP (FIG. 8). In striking contrast, both the full length BASP1-GFP fusion (“BASP1”) and the first 30 amino acids of BASP1 fused to GFP (“BASP1 (aa1-30)”) resulted in much greater GFP loading compared to CD81-GFP (“CD81”) or PDGFR-GFP (pDisplay”) (FIG. 9). These results suggest that BASP1 (full length or N-terminus) and full-length MARCKSL1 may be suitable scaffolds for luminal expression of exosomal cargo proteins.

### Example 4: Identification of a Minimal Protein Sequence Sufficient for Loading Luminal Exosome Payloads

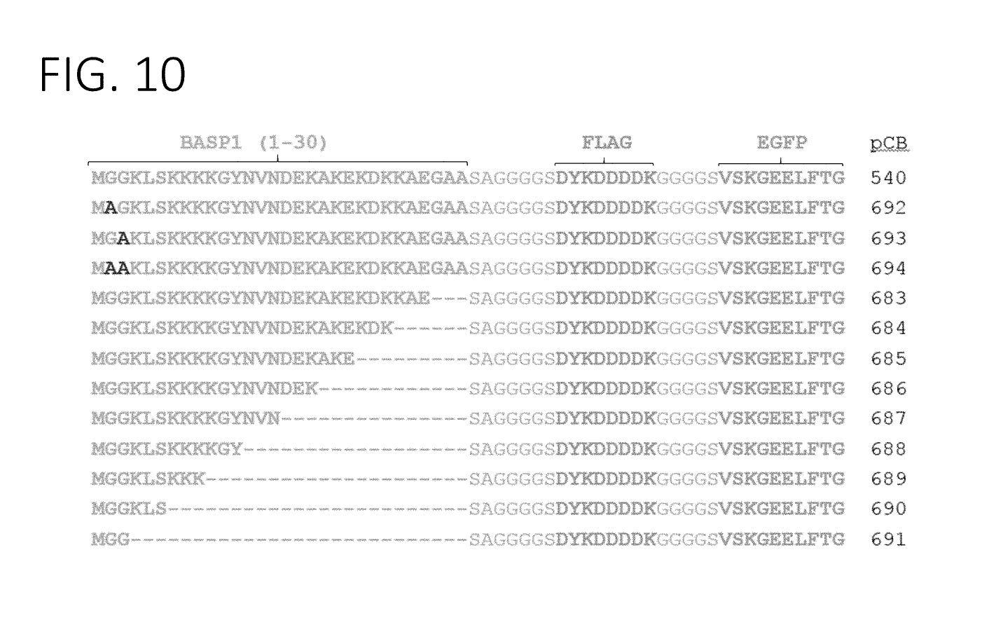

The results in Example 3 suggest that the N-terminal sequence of BASP1 is sufficient to load protein cargo into the lumen of exosomes. To determine the minimal peptide sequence with this activity, engineered GFP loading experiments were carried out by generating a variety of BASP1 truncations fused to the N-terminus of GFP and measuring the degree of their loading into exosomes. FIG. 10 shows the series of fusion proteins used in this experiment, indicating fragments and modifications of BASP 1 sequence, a FLAG tag for Western blotting detection, the first several amino acids of GFP, and glycine/serine linkers between each of the regions.

BASP1 has been reported to be myristoylated, which may play a role in its localization to the exosome lumen. To test the role of myristolyation in BASP1 loading, glycine to alanine point mutations at predicted myristolyation sites were also tested in the GFP loading experiments. Single mutations at position 2 (sequence pCB 692), position 3 (pCB 693), or a double mutation (pCB 694) were included with BASP1 1-30 (pCB 540) and tested with fusion proteins containing various truncations of BASP1 (pCB 683-691).

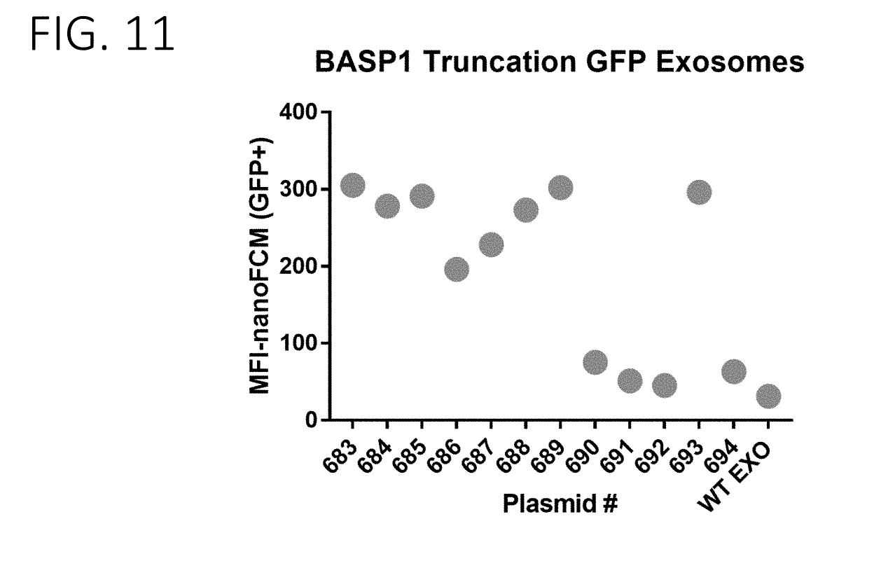

HEK293 SF cells were transfected and selected in the presence of puromycin to stably express the plasmids encoding each of the sequences in FIG. 10, and exosomes were purified as described in Example 1. The purified exosomes were analyzed for GFP fluorescence by nano-flow cytometry (Flow NanoAnlyzer, NanoFCM, Inc.) to determine the extent of GFP loading. As shown in FIG. 11, exosomes from untransfected cells (i.e., lacking GFP) showed very low signal (WT EXO). Exosomes containing BASP1 G2A-GFP (pCB 692) or BASP1 G2A/G3A-GFP (pCB 694) showed similarly low levels of GFP signal, while BASP1 G3A-GFP (pCB 693) showed much higher levels, indicating that the glycine in position two of BASP1 is essential for loading BASP1 fragments into exosomes, perhaps due to myristoylation. BASP1 truncations pCB683-689 also showed high levels of GFP signal, while shorter fragments pCB690-691 were similar to WT EXO. These results demonstrate that pCB689, a nine-amino acid fragment, is sufficient to drive protein cargo into the lumen of exosomes at a very high level.

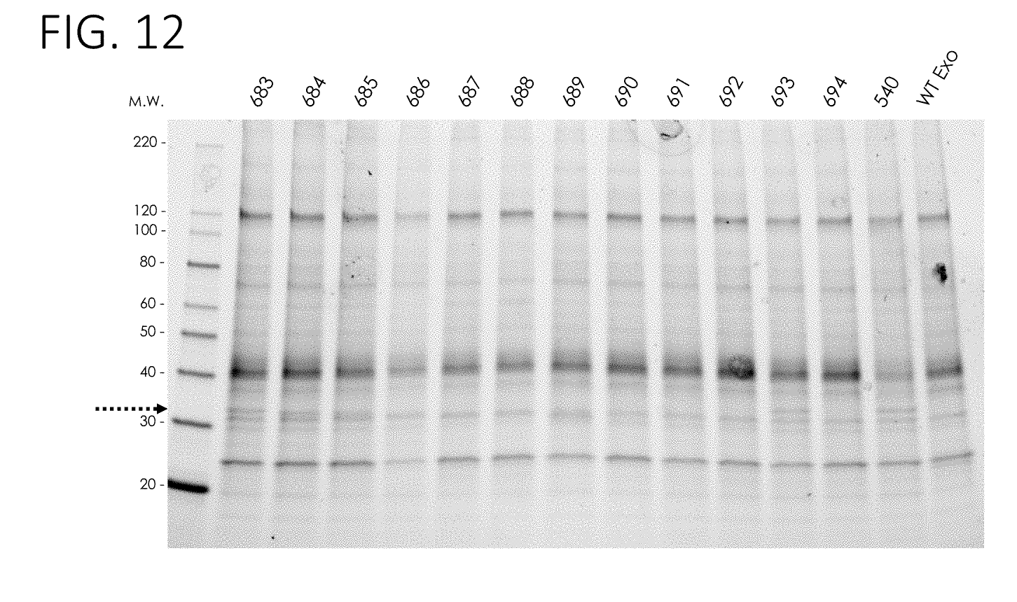

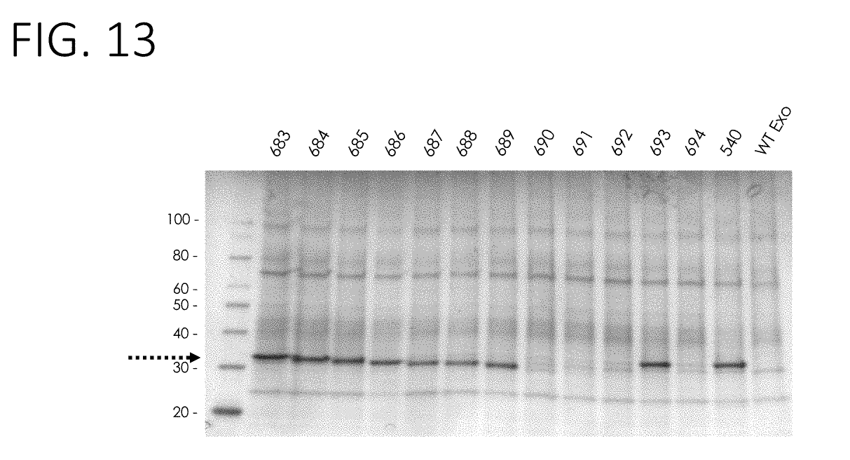

To confirm the results shown in FIG. 11, the BASP1 fragment-GFP exosomes were analyzed by protein blotting. Equal amounts of protein were loaded on an SDS-PAGE mini-PROTEAN® TGX Stain-Free Gel (Bio-Rad, Inc.) to measure total exosome protein (FIG. 12). The BASP1-GFP fragments were detectable in several lanes of the protein gel at ˜30 kDa (dotted arrow). This visualization method relies on the binding of a fluorescent molecule in the gel to tryptophan residues of the protein, yet there is only a single tryptophan residue in each of the BASP1-GFP fusion proteins, perhaps underestimating the abundance of BASP1 fragment in each lane. To achieve an unbiased measure of BASP1-GFP loading into exosomes, the protein gel containing the exosome samples was stained with Coomassie Blue (Invitrogen SimplyBlue SafeStain) (FIG. 13). The band pattern of the stained gel allowed for the clear identification of the BASP1-GFP fusion proteins (dotted arrow) and confirmed equal amounts of input protein in each sample, correlating with the results shown in FIG. 12.

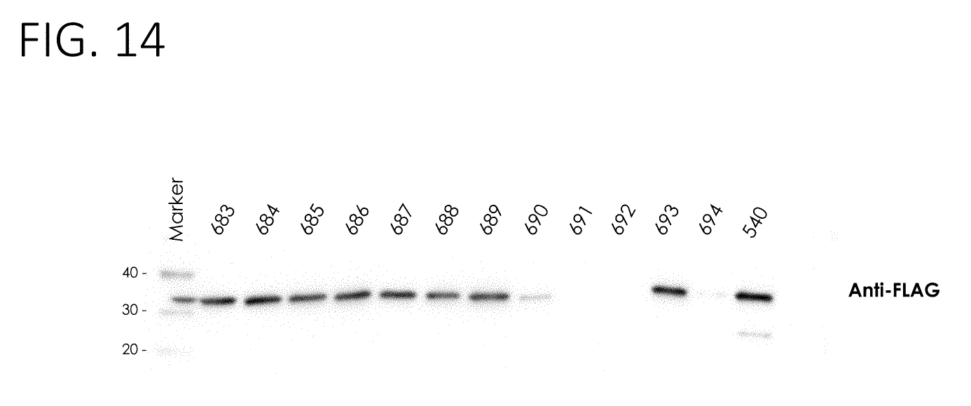

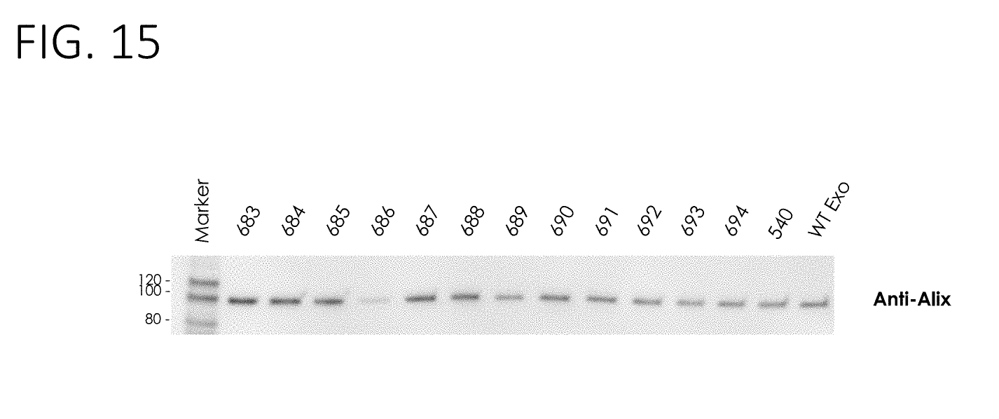

Western blotting with an anti-FLAG antibody (M2 monoclonal antibody, Millipore-Sigma) showed equal amounts of BASP1-GFP in pCB540 (amino acids 1-30) and pCB683-689 (FIG. 14), further showing the ability of BASP1 to load luminal exosome cargo. Anti-FLAG signal for the shorter fragments pCB690-691 was significantly reduced or absent. BASP1 G2A-GFP (pCB 692) or BASP1 G2A/G3A-GFP (pCB 694) also lacked signal, while BASP1 G3A-GFP (pCB 693) was expressed at levels similar to pCB540. These results agree with the nano-flow cytometry data in FIG. 11 and confirm that pCB689 is sufficient to load exosomes with protein cargo. Western blotting with an antibody against Alix, an established exosome protein, showed equal signal across all samples, indicating that BASP1-GFP overexpression did not disrupt the expression pattern of endogenous exosome proteins or otherwise disrupt exosome biogenesis or composition (FIG. 15). Together these results demonstrate that a nine-amino acid tag (MGGKLSKKK-SEQ ID NO: 13) can be expressed as a fusion to heterologous proteins and drive the localization of the protein into the lumen of exosomes. Additionally, position two of the sequence is required for exosome loading while position three of the sequence is dispensable. Thus, the sequences MGAKLSKKK (SEQ ID NO: 110) or, more generally, MGXKLSKKK (SEQ ID NO: 116) can also be used for loading any protein of interest into the exosome lumen.

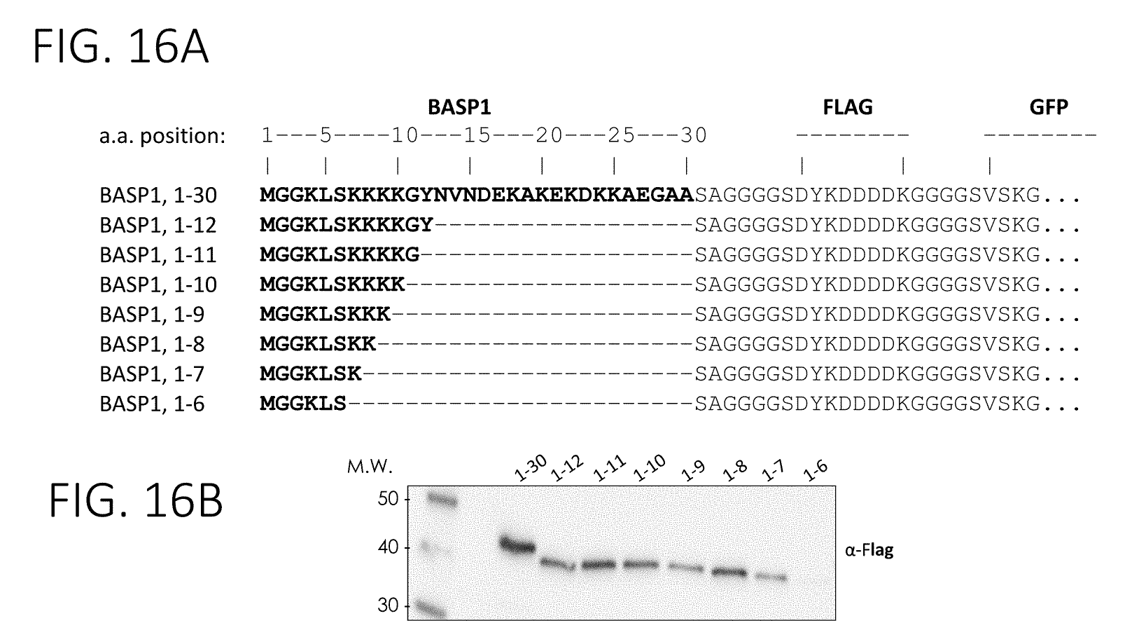

To identify the minimal BASP1 amino acid sequence between the twelve-amino acid truncation that facilitated loading and the six-amino acid truncation that failed to facilitate loading as shown above, individual truncation mutants of the N-terminus of BASP1 fused to a FLAG tag and GFP were generated and stably expressed in HEK293 SF cells (FIG. 16A). Exosomes were purified from the stable cell cultures as described above. BASP1 sequences of seven through twelve amino acids were capable of loading GFP in exosomes at high density, while the first six amino acids were not (FIG. 16B). These data demonstrate that at least one lysine residue after position six is required for luminal loading of exosomes with the N-terminus of BASP1.

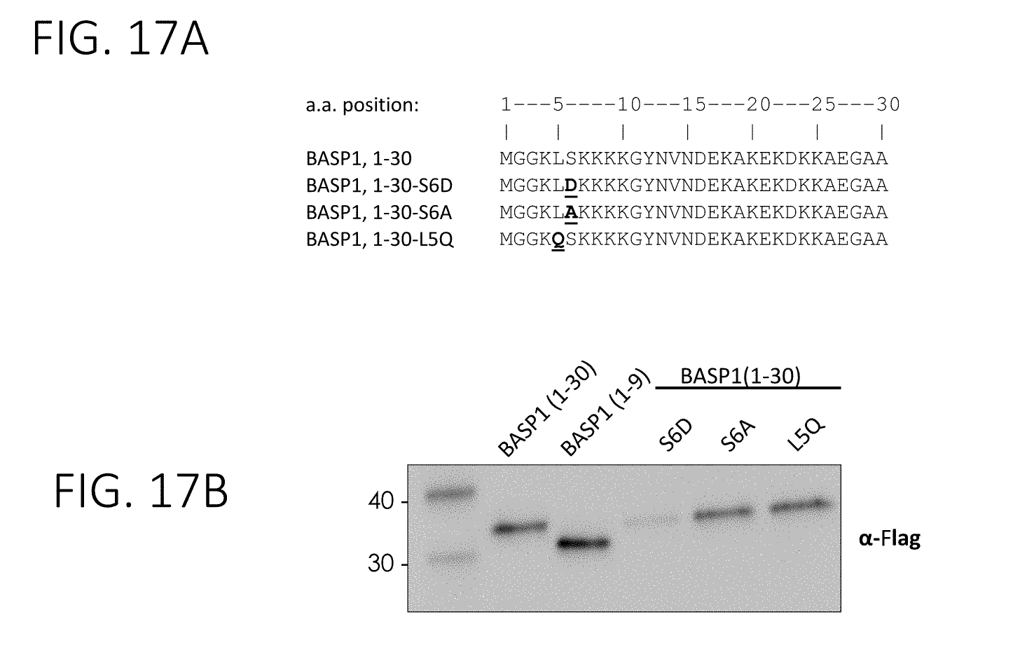

The serine at position 6 of BASP1 is highly conserved across species and in MARCKS and MARCKSL1. To determine whether this amino acid was required for cargo loading into exosomes, HEK293SF cells were stably transfected with expression plasmids encoding BASP1 1-30-FLAG-GFP or BASP1 1-30-FLAG-GFP including a point mutant, replacing serine six with aspartic acid (S6D; polar charged substitution) or alanine (S6A; small nonpolar substitution). Additionally, the lysine at position five was mutated to a glutamic acid (L5Q) to test the potential role of this position in modulating myristoylation, palmitoylation, and other membrane functions of several membrane-associated proteins (Gottlieb-Abraham et al., Mol. Biol. Cell. 2016 Dec. 1; 27(24):3926-3936) (FIG. 17A). BASP1 S6D completely abrogated loading of GFP into exosomes, while S6A did not alter loading. BASP1 L5Q did not impact luminal loading either, indicating that a negative charge at position six disrupts loading, while a polar amino acid substitution at position five is well-tolerated (FIG. 17B).

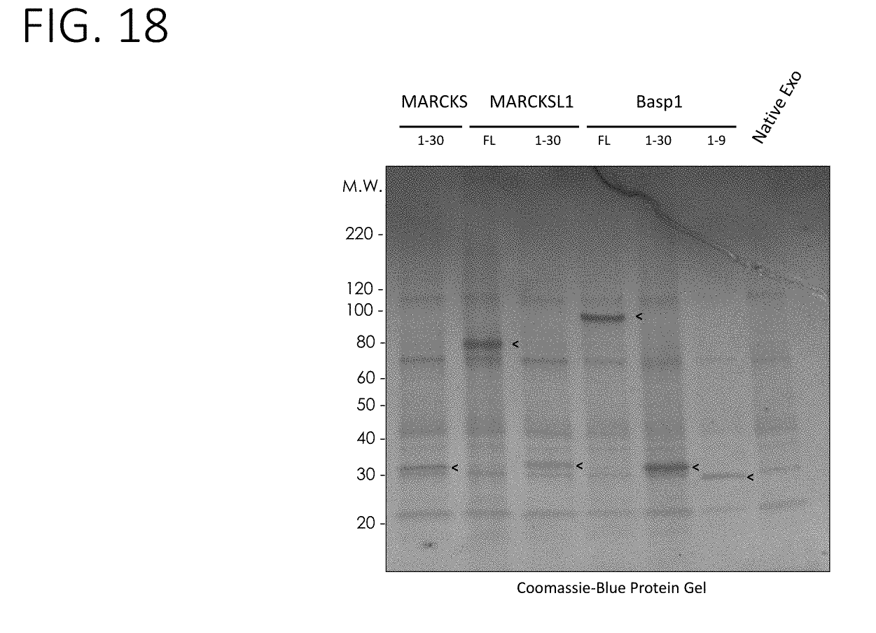

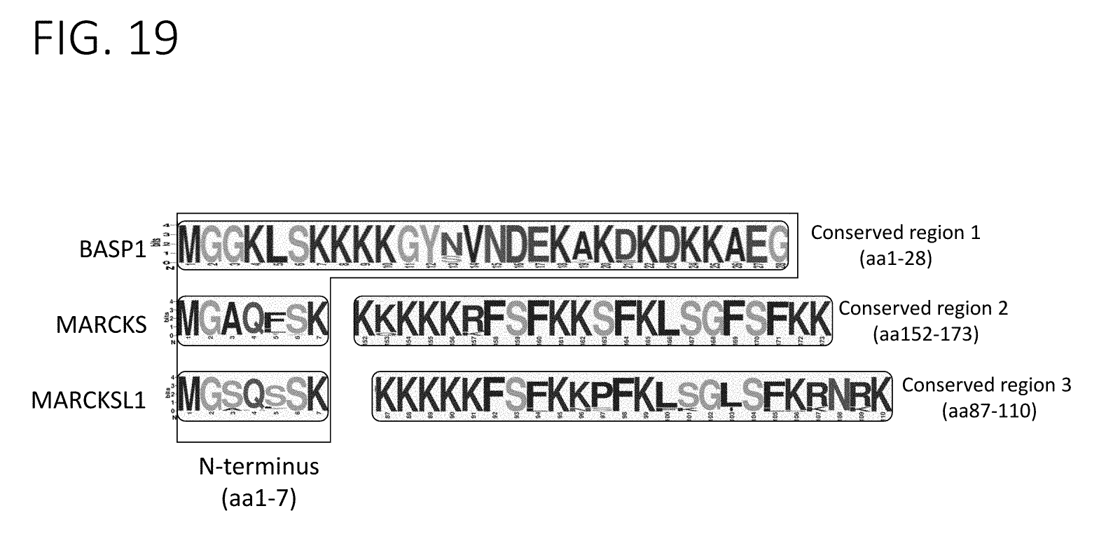

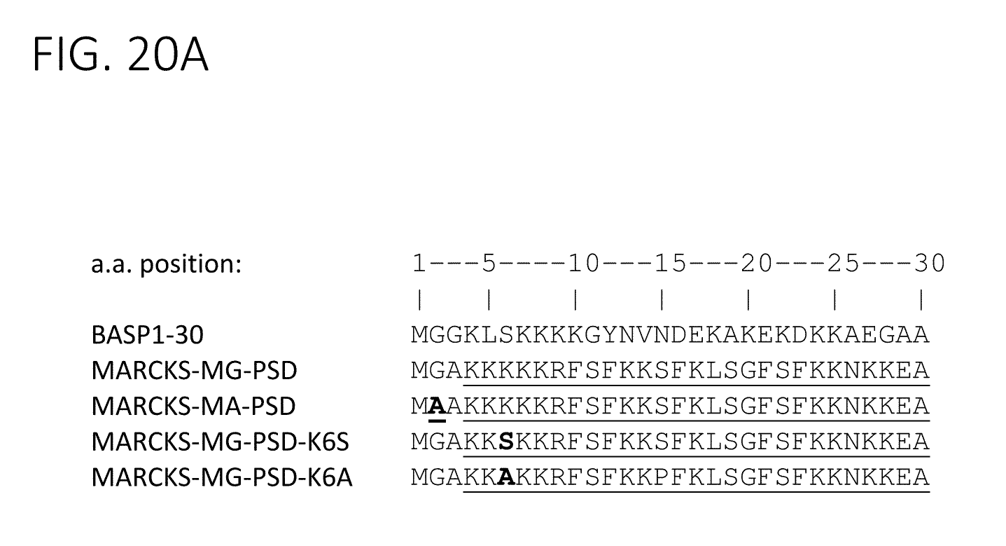

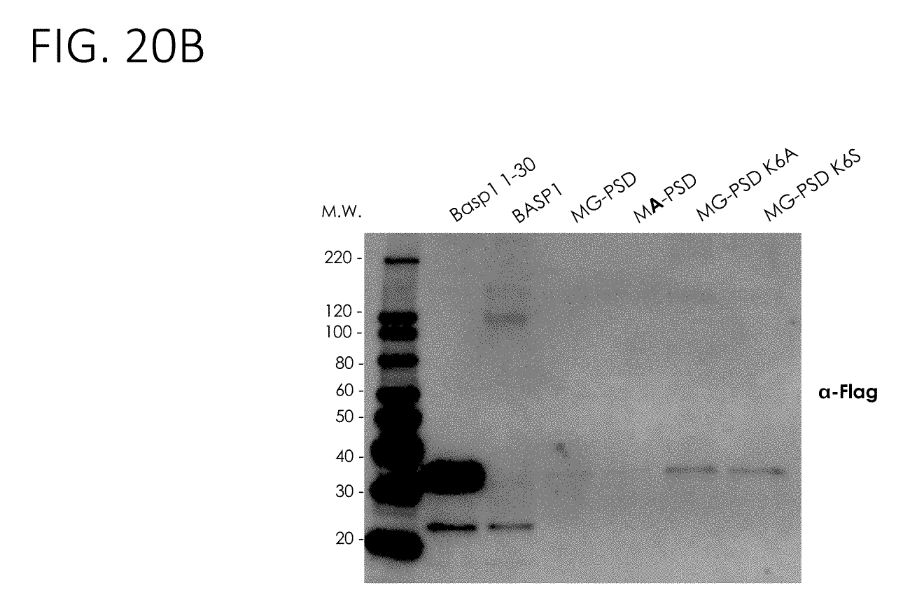

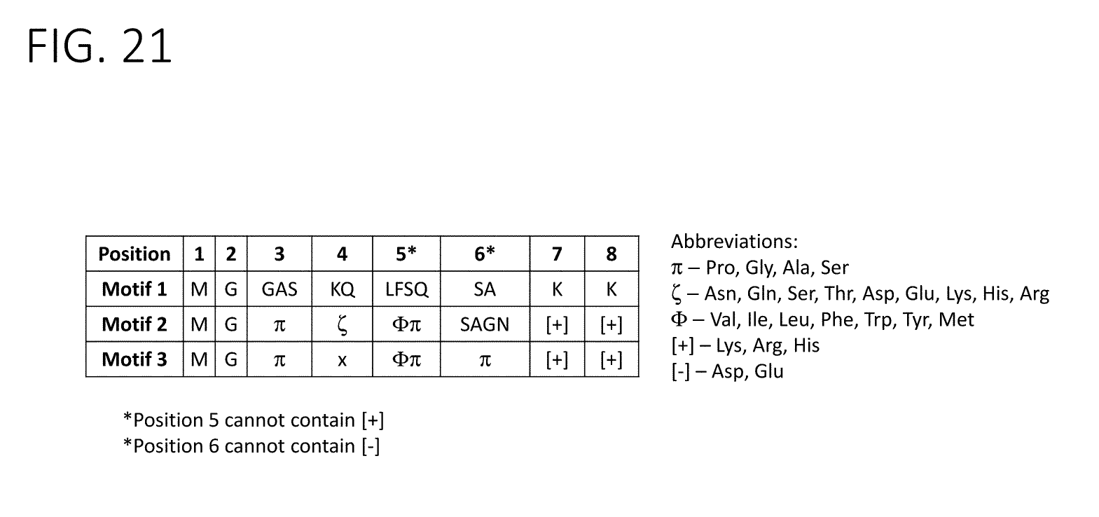

The first thirty amino acids of BASP1 contain the N-terminal leader sequence identified above, followed by a lysine-rich stretch of amino acids. To understand whether MARCKS and MARCKSL1 N-termini can load exosomes similarly to BASP1, HEK293SF cells were stably transfected with MARCKS and MARCKSL1 full-length proteins or amino acids 1-30 fused to FLAG-GFP. Purified exosomes were analyzed by SDS PAGE and Coomassie staining to determine the extent of loading. Full-length MARCKS and MARCKSL1 were able to load exosomes with GFP, but amino acids 1-30 were inferior to the full-length proteins, suggesting that there are additional structural or sequence features in distal regions of the MARCKS and MARCKSL1 proteins required for loading (FIG. 18). Sequence analysis of MARCKS and MARCKSL1 revealed regions with potential sequence homology to the N-terminus of BASP1. Amino acids 152-173 of MARCKS and 87-110 of MARCKSL1 are lysine-rich with interspersed phenylalanine and serine residues and are predicted to be phosphorylation site domains (PSD) or effector domains (ED) (FIG. 19). HEK293 SF cells were stably transfected with plasmid constructs fusing amino acids 1-3 of MARCKS to the PSD domain (MG-PSD). Individual point mutations were generated at the predicted myristoylation site (MA-PSD) and position six (K6S and K6A) to determine the role of these residues in loading exosomes (FIG. 20A). Western blotting of purified exosomes demonstrated that compared to the positive control of BASP1 1-30, neither MG-PSD nor MA-PSD could efficiently load exosomes. Interestingly, the K6A and K6S mutations led to improvements in loading, suggesting that a positive charge at position 6 prevents loading of exosomal cargo and that the PSD of MARCKS could functionally complement for the endogenous N-terminal sequence (FIG. 20B). Together, these studies allowed for the identification of several motifs sufficient to load cargo into exosomes (FIG. 21).

The narrowest motif, Motif 1, allows for a protein sequence of (M)(G)(G/A/S)(K/Q)(L/F/S/Q)(S/A)(K)(K) (SEQ ID NO: 118), where each parenthetical letter or group of letters is an amino acid position, and wherein additionally position five cannot be a positively charged amino acid (K/R/H) and position six cannot be a negatively charged amino acid (D/E). Sub-motifs of Motif 1 include, without being limiting, the protein sequences: (M)(G)(G)(K/Q)(L/F/S/Q)(S/A)(K)(K), (M)(G)(A)(K/Q)(L/F/S/Q)(S/A)(K)(K), (M)(G)(S)(K/Q)(L/F/S/Q)(S/A)(K)(K), (M)(G)(G/A/S)(K)(L/F/S/Q)(S/A)(K)(K), (M)(G)(G/A/S)(Q)(L/F/S/Q)(S/A)(K)(K), (M)(G)(G/A/S)(K/Q)(L)(S/A)(K)(K), (M)(G)(G/A/S)(K/Q)(F)(S/A)(K)(K), (M)(G)(G/A/S)(K/Q)(S)(S/A)(K)(K), (M)(G)(G/A/S)(K/Q)(Q)(S/A)(K)(K), (M)(G)(G/A/S)(K/Q)(L/F/S/Q)(S)(K)(K) and (M)(G)(G/A/S)(K/Q)(L/F/S/Q)(A)(K)(K), where position five cannot be a positively charged amino acid (K/R/H) and position six cannot be a negatively charged amino acid (D/E).

Motif 2, a broader motif, can be expressed as (M)(G)(π)(ξ)(Φ/π)(S/A/G/N)(+)(+), wherein each parenthetical position represents an amino acid, and wherein π is any amino acid selected from the group consisting of (Pro, Gly, Ala, Ser), ξ is any amino acid selected from the group consisting of (Asn, Gln, Ser, Thr, Asp, Glu, Lys, His, Arg), Φ is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu).

Motif 3, the broadest motif, can be expressed as (M)(G)(π)(X)(Φ/π)(π)(+)(+), wherein each parenthetical position represents an amino acid, and wherein π is any amino acid selected from the group consisting of (Pro, Gly, Ala, Ser), X is any amino acid, 41) is any amino acid selected from the group consisting of (Val, Ile, Leu, Phe, Trp, Tyr, Met), and (+) is any amino acid selected from the group consisting of (Lys, Arg, His); and wherein position five is not (+) and position six is neither (+) nor (Asp or Glu). In all cases of Motifs 1-3, the sequence may be truncated by one amino acid to be seven total amino acids in length (i.e., consisting of amino acids 1-7 in the order presented in Motifs 1-3). Any of the sequences derived from any of Motifs 1, 2, or 3 (or these motifs lacking amino acid 7), can be used to load cargo into exosomes to the same extent as, or comparable to, full length BASP1 or natural truncation sequences of BASP1. This deep analysis of amino acid sequence-structure-function provides novel insights into the requirements for directing biologically expressed cargo into exosomes by producer cells.

### Example 5: The N-Terminus of BASP1 is Sufficient to Load Diverse Classes of Proteins

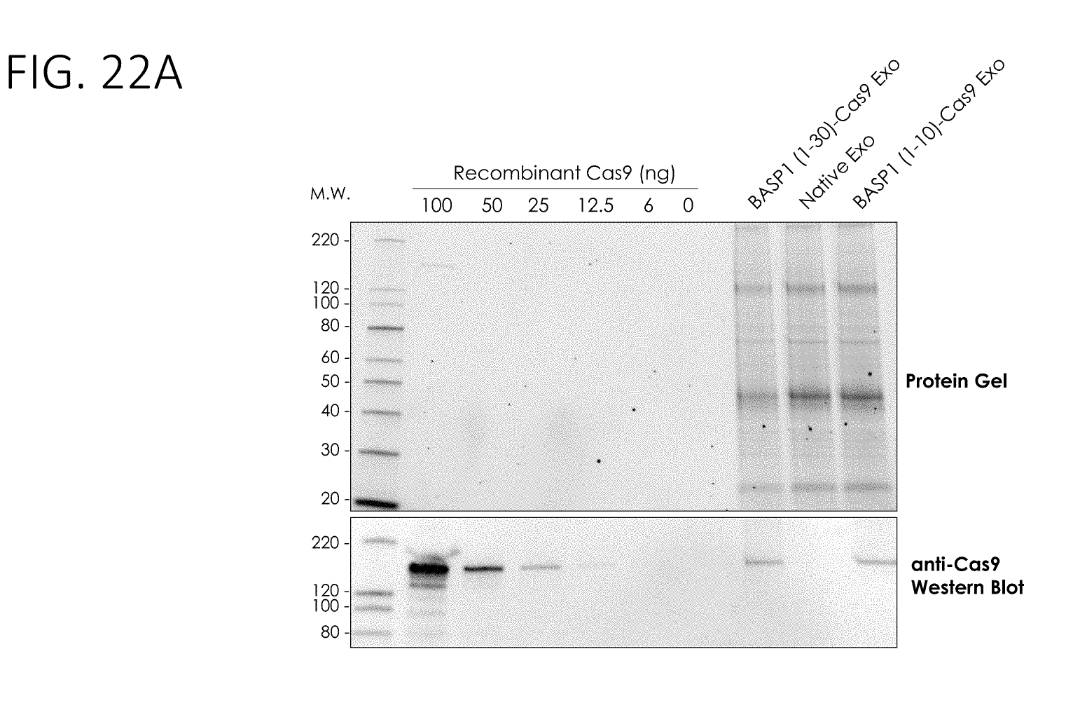

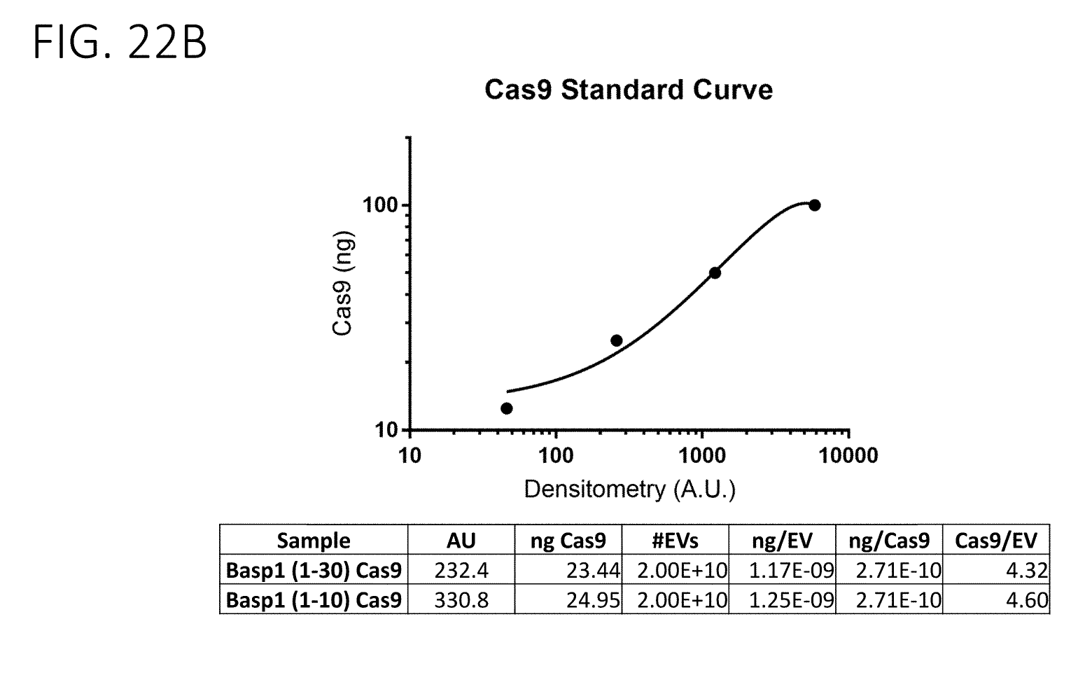

The results in Example 4 suggest that the N-terminus of BASP1 may be a useful engineering scaffold for generating luminally loaded exosomes directly from producer cells. To test this hypothesis, stable HEK293 SF cells were generated to express full-length Cas9 protein with codon optimization (as described in Zetsche B, Volz S E, Zhang F. A split-Cas9 architecture for inducible genome editing and transcription modulation. Nat Biotechnol. 2015 February; 33(2):139-42) fused to amino acids 1-30 or 1-10 of BASP1. Exosomes were purified from cell culture as described above and analyzed by SDS-PAGE and Western blotting using an anti-Cas9 antibody (Abcam; Catalog # ab191468, clone 7A9-3A3). As shown in FIG. 22A, both BASP1 1-30 and 1-10 were sufficient to load Cas9 in exosomes. Recombinant Cas9 protein was used as a positive control for Western blotting. Densitometry quantitation and comparison of various amounts of recombinant Cas9 and BASP1-Cas9 exosome lanes from the Western blotting experiments revealed that the exosomes were loaded with 4-5 Cas9 molecules per exosome (FIG. 22B). This Cas9 enzyme, which is ˜160 kDa in mass, represents a significant increase in cargo size compared to the GFP experiments shown above.

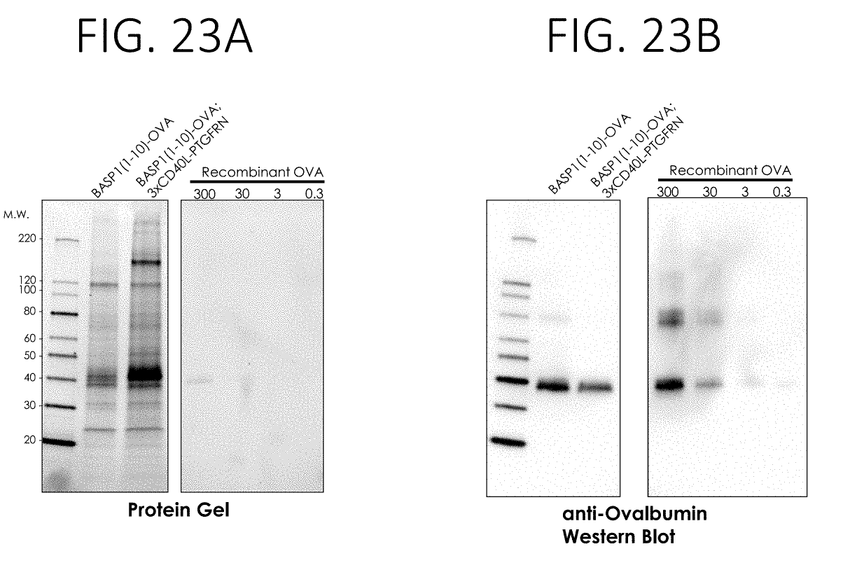

As an additional validation of the diversity of cargo proteins that can be loaded as a fusion to the N-terminus of BASP1, ovalbumin was stably expressed in HEK293SF cells as a fusion to amino acids 1-10 of BASP1 (“BASP1(1-10)-OVA”). A separate cell line was co-transfected with the same plasmid and a second plasmid encoding trimeric CD40L fused to an exosome-specific surface glycoprotein PTGFRN (“3xCD40L-PTGFRN”) using a second selectable marker. Exosomes were purified from the two transfected cell cultures and analyzed by SDS-PAGE (FIG. 23A) and anti-ovalbumin western blotting (Abcam; Catalog # ab17293, clone 6C8) (FIG. 23B). As a control, recombinant ovalbumin (InvivoGen; Catalog # vac-pova) was titrated in a separate gel. Ovalbumin was robustly loaded into exosomes when fused to amino acids 1-10 of BASP1 as a single construct or when in combination with an additional overexpression plasmid (3xCD40L-PTGFRN). This result demonstrates that exosomes can be combinatorially engineered, both with luminal cargo and with a simultaneous surface cargo (e.g., PTGFRN) from a separate transcript.

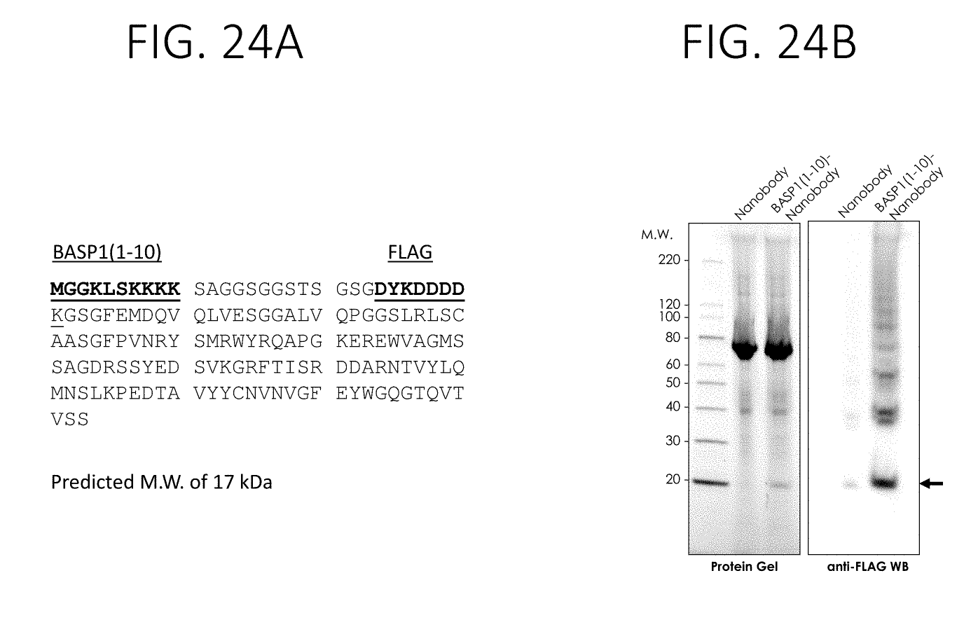

Another class of proteins that may be useful in the context of therapeutic exosomes are antibodies and antibody fragments. A single chain camelid nanobody targeting GFP (as described in Caussinus E, Kanca O, Affolter M. Fluorescent fusion protein knockout mediated by anti-GFP nanobody. Nat Struct Mol Biol. 2011 Dec. 11; 19(1):117-21) was stably expressed in HEK293SF cells as a fusion protein to amino acids 1-10 of BASP1 and a FLAG tag (“BASP1(1-10)-Nanobody”) or a FLAG tag alone (“Nanobody”) (FIG. 24A). Purified exosomes were analyzed by SDS-PAGE and anti-FLAG Western blotting, demonstrating that there was substantial enrichment of the nanobody with equal amounts of total loaded protein when the nanobody was fused to the N-terminus of BASP1 (FIG. 24B). These results demonstrate that protein cargo of diverse classes can be expressed and packaged into exosomes by producer cells using a very short protein sequence derived from the N-terminus of BASP1.

### Example 6: The N-Terminus of BASP1 Can Be Used to Load Nucleic

Acids in the Lumen of Exosomes

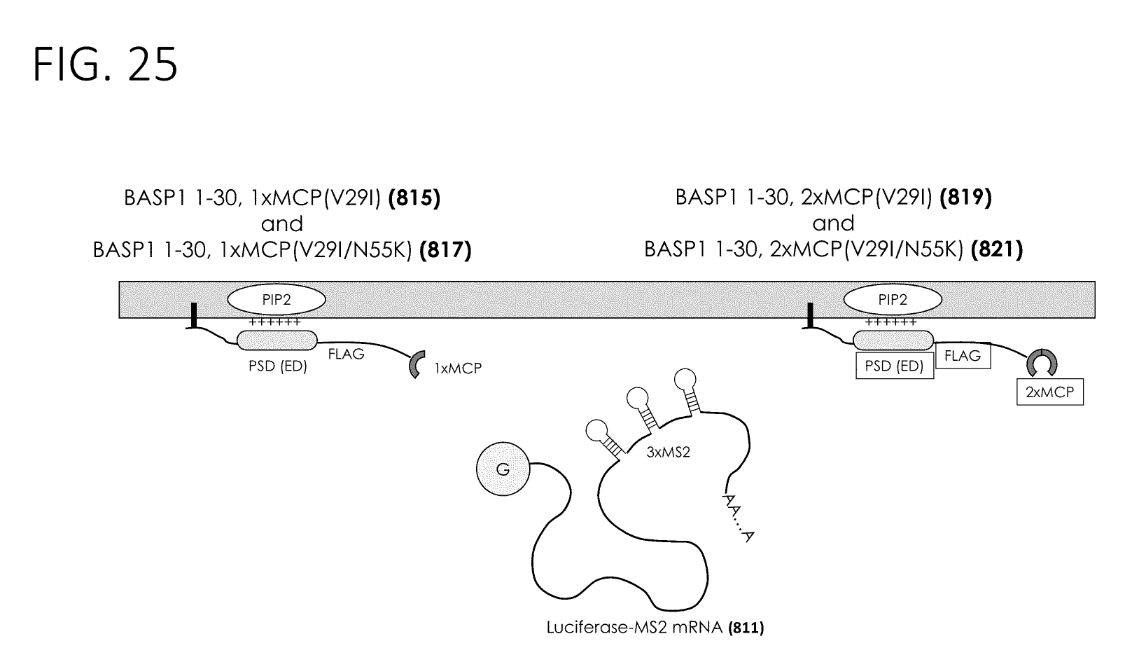

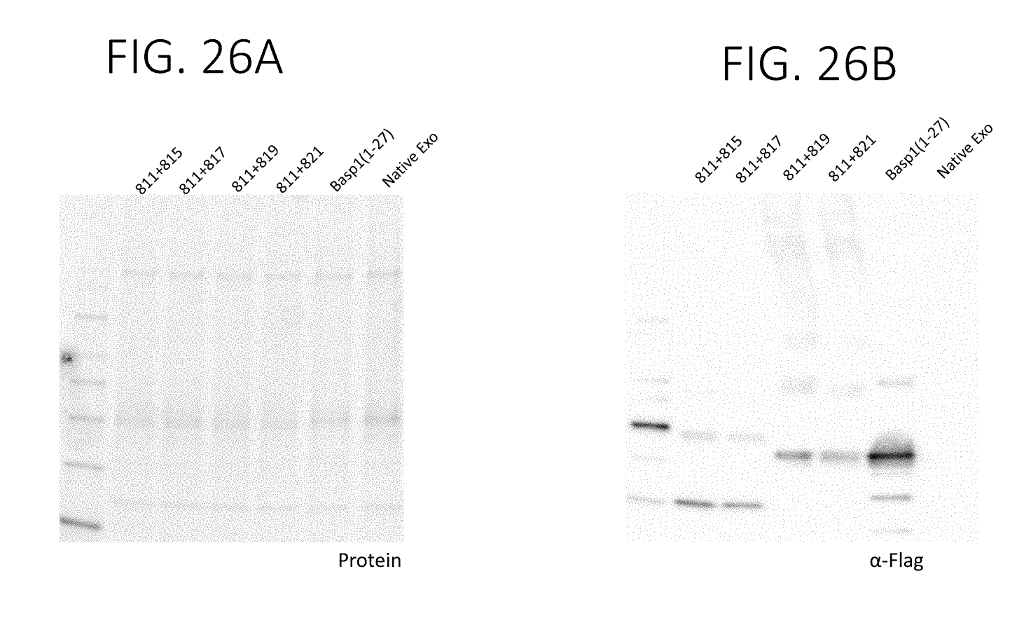

Nucleic acids, and in particular RNAs (e.g., mRNAs, siRNAs, miRNAs) are an attractive class of therapeutic cargo to be loaded in the lumen of therapeutic exosomes. Exosome loading of RNA may protect the RNA from degradation in the extracellular environment and the loaded exosome can be directed to certain cells and/or tissues through additional levels of exosome engineering, e.g., surface expression of a targeting construct. To understand whether the exosome proteins (or protein fragments) identified above can be used to generate mRNA-loaded exosomes, combinatorial engineered exosomes were generated. As shown in FIG. 25, amino acids 1-30 of BASP1 were expressed as a fusion to FLAG and variants of the phage protein MCP. MCP recognizes and binds to an mRNA stem loop called MS2, which can be expressed as a transcriptional fusion to mRNAs and other RNAs, thus driving physical association between the MCP fusion proteins and MS2 fusion RNAs of interest. Mutational analysis previously identified two positions in MCP that increases affinity to MS2; a valine to isoleucine substitution at position 29 (V29I; Lim & Peabody, RNA. Nucleic Acids Res. 1994 Sep. 11; 22(18):3748-52) and an asparagine to lysine substitution at position 55 (N55K; Lim et al., J Biol Chem. 1994 March 25; 269(12):9006-10). BASP11-30 was fused to monomeric or dimeric MCP variants, where each MCP was either V29I or doubly mutated V29I/N55K. A luciferase reporter construct was expressed as a fusion to 3 MS2 stem loops from a separate plasmid. Five stable HEK293SF cell lines were generated, either Luciferase-MS2 alone (#811) or in combination with each of the BASP1-MCP variants (#815, 817, 819, or 821) (FIG. 25). As an additional control, HEK293SF cells were stably transfected with FLAG-tagged BASP1 1-27. Exosomes were isolated and treated with Benzonase® to remove any externally-associated mRNAs, and purified according to the Methods above. Purified exosomes were analyzed by SDS-PAGE (FIG. 26A) and anti-FLAG Western blotting (FIG. 26B), demonstrating equal amounts of total protein and comparable levels of BASP1-FLAG fusions in each exosome preparation. Importantly, the BASP1-MCP fusions expressed to comparable levels as a BASP1 1-27 FLAG fusion lacking an MCP protein, demonstrating that the addition of MCP monomers or dimers do not disrupt the BASP1-mediated loading of proteins in exosomes.

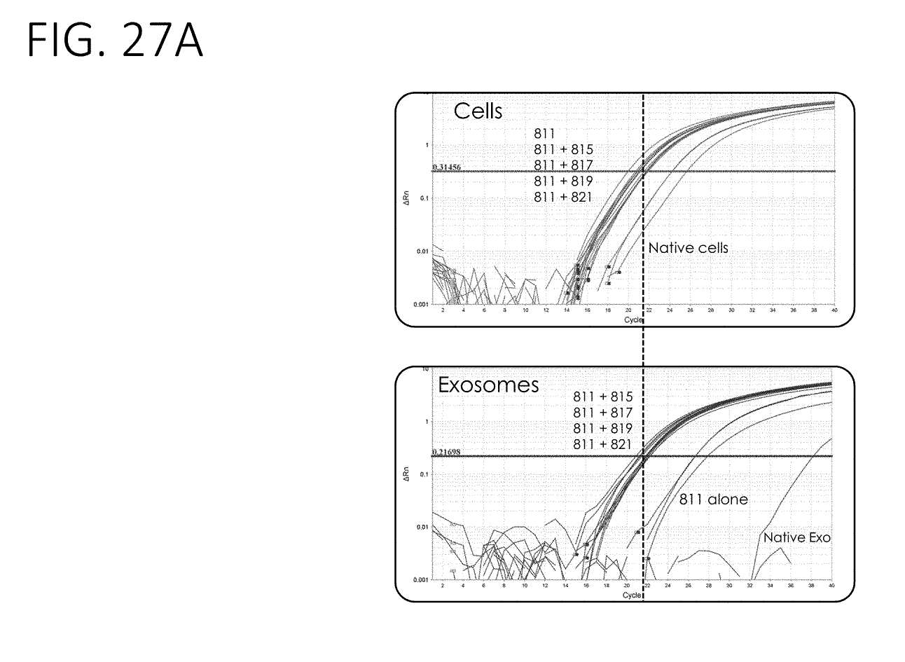

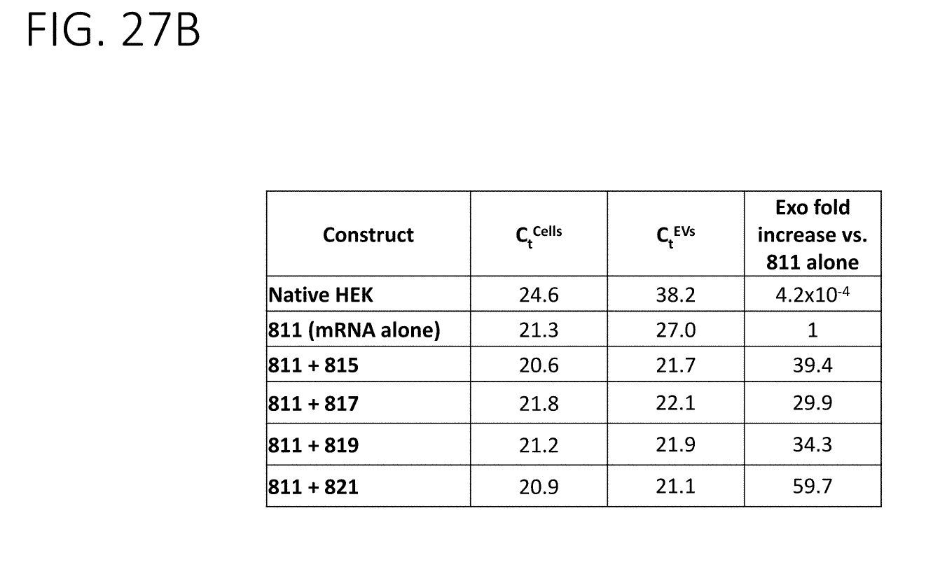

The cells stably expressing the BASP1-MCP and Luciferase-MS2 mRNA were isolated and total Luciferase mRNA was quantified by RT-qPCR (FWD Primer: 5′-TGGAGGTGCTCAAAGAGTTG-3′ (SEQ ID NO: 119); REV Primer: 5′-TTGGGCGTGCACTTGAT-3′ (SEQ ID NO: 120); PROBE: 5′-/56-FAM/CAGCTTTCC/ZEN/GGGCATTGGCTTC/3IABkFQ/-3′ (SEQ ID NO: 121)). Untransfected cells expressed lower levels of Luciferase than all of the 811-expressing cells, which expressed comparable levels of Luciferase (FIG. 27A, top). The purified exosomes from each of the stable cell lines were also analyzed by RT-qPCR. Native exosomes had no detectable levels of Luciferase MS2, while cells expressing 811 alone had detectable but very low levels of Luciferase MS2. Importantly, each of the BASP1-MCP fusion proteins contained greater amounts of Luciferase-MS2 mRNA, demonstrating the importance of the binding between MCP and MS2 to facilitate loading of mRNA into exosomes (FIG. 27A, bottom). Quantitation of relative mRNA between the groups demonstrated an enrichment of ˜30 to 60-fold for all of the BASP1-MCP fusions over 811 alone (FIG. 27B). BASP1-MCP construct 821, which contained dimeric MCP V29I/N55K is predicted to have the greatest affinity for MS2 mRNAs, and indeed contained the greatest amount of Luciferase-MS2 in this experiment. These results demonstrate that BASP1 fragments are robust and versatile scaffold proteins for loading the lumen of exosomes with diverse cargo including nucleic acids.

### Example 7: BASP1, MARCKS, and MARCKSL1 Can Be Used to

Generate Surface-Decorated Exosomes

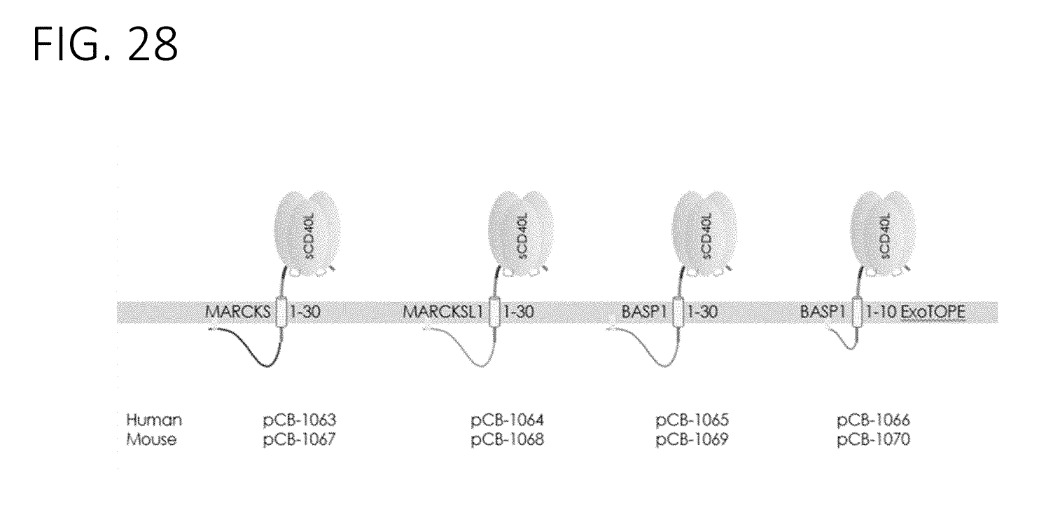

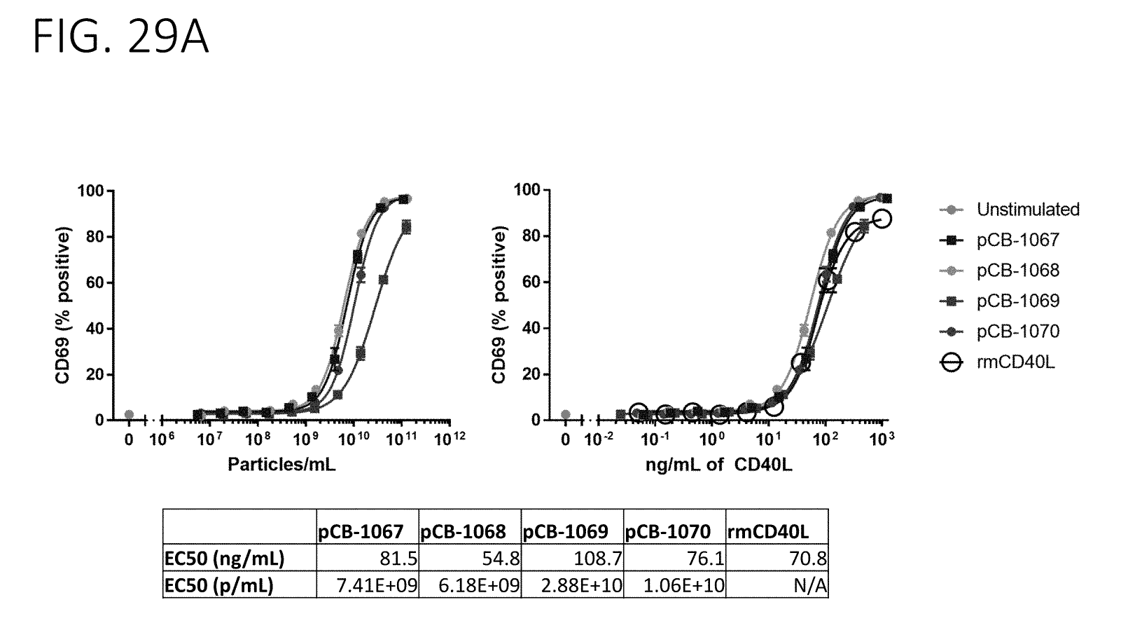

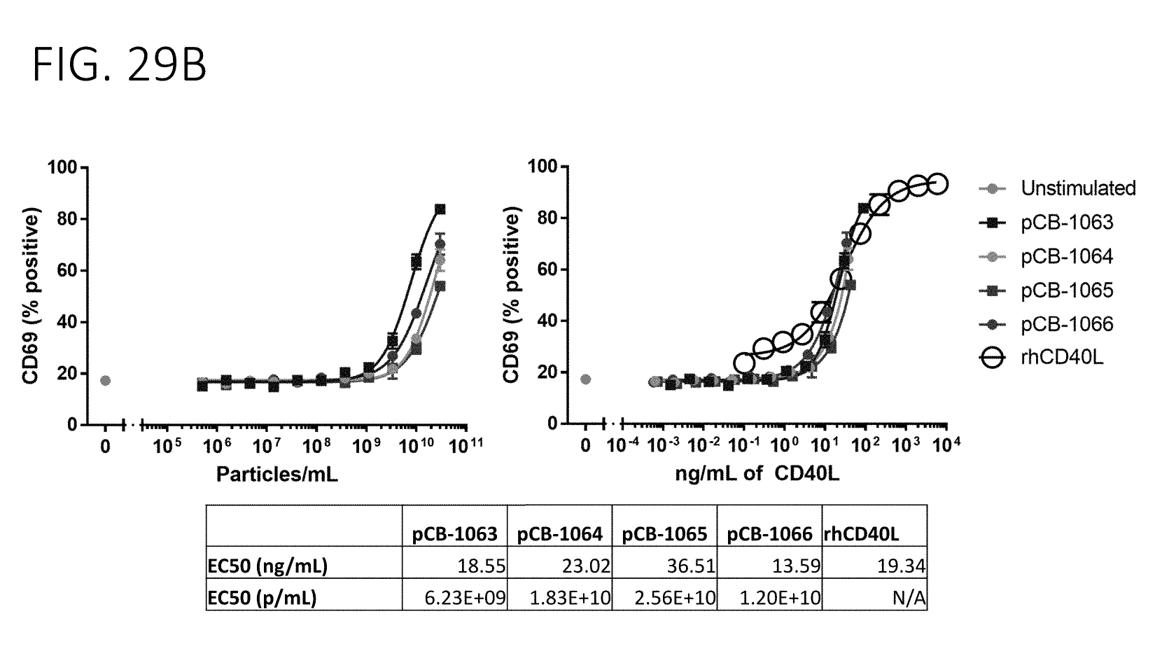

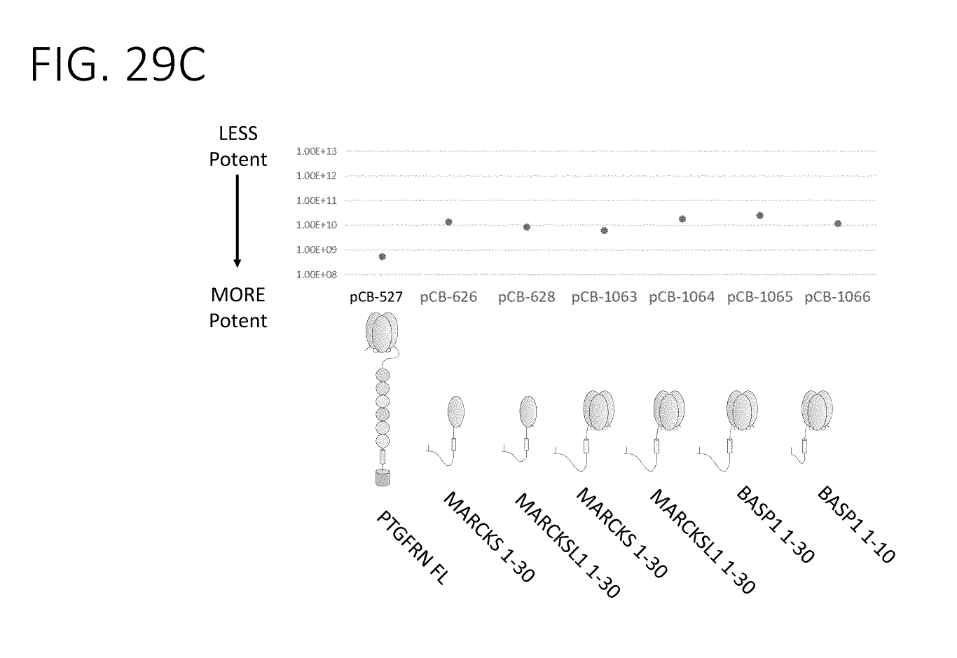

The results in the previous experiments demonstrate that full-length and N-terminal regions of MARCKS, MARCKSL1, and BASP1 can be used to generate luminally loaded exosomes. To further explore the potential of these proteins for exosome engineering, amino acids 1-30 of MARCKS, MARCKSL1 and BASP1, or amino acids 1-10 of BASP1 were fused to the endogenous transmembrane region of CD40L expressed as a homotrimer. Constructs were prepared for both human and mouse sequences of CD40L because the ligands do not cross-react with the cognate receptor on the other species (FIG. 28). Exosomes were purified from HEK293SF cells stably transfected with one of the CD40L expression constructs and incubated in either mouse or human B cells. Amounts of input CD40L on the exosomes was quantified by CD40L ELISA (for measurement of human CD40L, R&D Systems, Catalog # DCDL40, Lot # P168248; and for measurement of mouse CD40L, Abcam, Catalog # ab119517, Lot # GR3218850-2 were used), B cells were quantified using B-cell marker, CD19, and B cell activation was measured by percentage of gated cells positive for CD69. Dose titration curves of mouse (FIG. 29A) or human (FIG. 29B) exosomal CD40L in species-matched cultures showed comparable activity between constructs on a particle-to-particle basis (left graphs and table below) or as compared to each other and equal amounts of recombinant protein on a CD40L molar basis (right graphs and table below). Comparable activity was observed when the CD40L constructs were expressed as monomers as well, and were only slightly less potent than trimeric CD40L expressed on the N-terminus of PTGFRN, a high-density exosome display scaffold (see, e.g., International Patent Application No. PCT/US2018/048026) (FIG. 29C). These results demonstrate that MARCKS, MARCKSL1, and BASP1 are diverse, robust scaffolds useful for the generation of various classes of engineered exosomes for use in human and animal applications.

### Example 8: Diverse Cell Types Express BASP1, MARCKS, and/or MARCKSL1

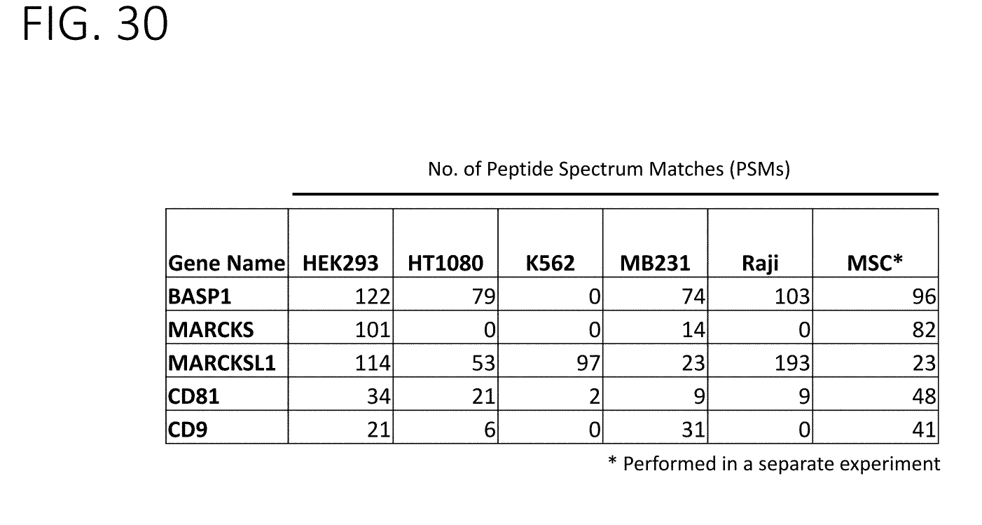

Cell lines from different tissues of origin (HEK293, kidney; HT1080, connective tissue; K562, bone marrow; MDA-MB-231, breast; Raji, lymphoblast) were grown to logarithmic phase and transferred to media supplemented with exosome-depleted serum for ˜6 days except for the HEK293 cells, which were grown in chemically-defined media. Bone marrow-derived mesenchymal stem cells (MSC) were grown on 3D microcarriers for five days and supplemented in serum-free media for three days. Supernatant from each cell line culture was isolated, and exosomes were purified using the Optiprep™ density-gradient ultracentrifugation method described above. Each of the purified exosome preparations was analyzed by LC-MS/MS as described above, and the number of peptide spectrum matches (PSMs) was quantified for BASP1, MARCKS, and MARCKSL1 and two widely studied exosome proteins (CD81 and CD9). The tetraspanins CD81 and CD9 were detectable in most purified exosome populations, but were, in some cases, equal to or lower than the luminal exosome proteins (e.g., compare CD9 to BASP1 or MARCKSL1) (FIG. 30). This finding indicates that the newly-identified luminal exosome markers may be suitable fusion proteins for generating engineered exosomes from several unrelated cell lines derived from different tissues.

### Example 9: Non-Human Cells Overexpressing BASP1 Produce

Luminally Engineered Exosomes

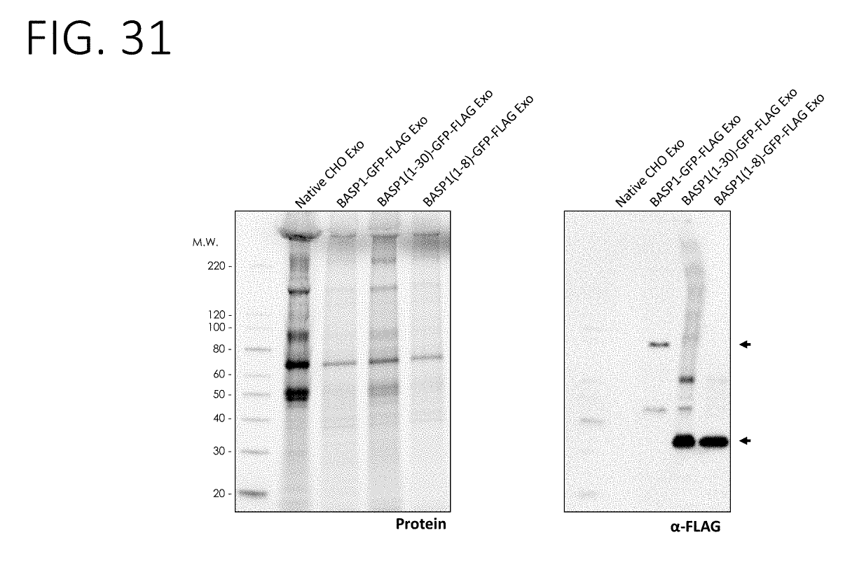

The results in Example 8 demonstrate that numerous human-derived cells naturally express BASP1 and the other novel exosome proteins identified in Example 1. To determine whether BASP1 can be used as a universal exosome scaffold protein, Chinese hamster ovary (CHO) cells were stably transfected with either a plasmid expressing full-length BASP1 fused to a FLAG tag and GFP (“BASP1-GFP-FLAG”), a plasmid expressing amino acids 1-30 of BASP1 fused to a FLAG tag and GFP (“BASP1(1-30)-GFP-FLAG”) or a plasmid expressing amino acids 1-8 of BASP1 fused to a FLAG tag and GFP (“BASP1(1-8)-GFP-FLAG”). Exosomes were purified from wild-type CHO cells and CHO cells transfected with one of the three BASP1 plasmids using the method described in Example 1. As shown in FIGS. 31A-B, BASP1 and the BASP1 fragment fusion proteins were successfully overexpressed in CHO cells and loaded into exosomes as detected by stain-free PAGE (FIG. 31A) and Western blotting with an antibody against FLAG (FIG. 31B). This result demonstrates that non-human cells, such CHO cells, can produce exosomes that overexpress human BASP1 fragments, and that this overexpression can drive a cargo protein into the lumen of exosomes at high density. This result indicates that BASP1 is a universal scaffold protein for generating engineered exosomes from many different cell types and species.

### Example 10: Generation of Lumen-Engineered Exosomes

A producer cell generating lumen-engineered exosomes is made by introducing an exogenous sequence encoding an exosome protein or a modification or a fragment of the exosome protein. The exosome protein is a fusion protein comprising the BASP1 fragments disclosed in Example 4 above, and a cargo protein. A plasmid encoding an exosome protein is transiently transfected to induce high-level expression of the exosome protein in the exosome lumen.

A polynucleotide encoding an exosome protein, a modification or a fragment of an exosome protein, or an exogenous sequence encoding a therapeutic peptide, cargo peptide, or a targeting moiety is stably transformed into a producer cell to produce lumen-engineered exosomes. The exogenous sequence encoding a therapeutic peptide, cargo peptide, or a targeting moiety is inserted into a genomic site encoding an exosome protein to generate a fusion protein comprising the therapeutic peptide or cargo peptide attached to the exosome protein. A polynucleotide encoding a modified exosome protein is knocked in to a genomic site encoding an exosome protein.

A producer cell line is generated by stably transfecting at least two polynucleotides, each encoding an exosome protein, a modification or a fragment of an exosome protein, or an exogenous peptide (e.g., targeting moiety, therapeutic peptide). Two or more exogenous sequences are inserted into multiple genomic sites, within or closed to the genomic sequence encoding an exosome protein, to generate a lumen-engineered exosome comprising multiple modified exosome proteins. Each of the plurality of modified exosome proteins is targeted to the lumen of exosomes.

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

The present disclosure provides, inter alia, compositions of exosomes containing modified exogenous proteins and peptides for use in enrichment of exogenous proteins in exosomes. The present disclosure also provides method of and methods of producing enriched exosomes. While various specific embodiments have been illustrated and described, the above specification is not restrictive. It will be appreciated that various changes can be made without departing from the spirit and scope of the invention(s). Many variations will become apparent to those skilled in the art upon review of this specification.

1. An exosome comprising a target protein, wherein at least a part of the target protein is expressed from an exogenous sequence, and the target protein comprises BASP1 or a fragment thereof.

2. The exosome of claim 1, wherein the target protein is present in the lumen of the exosome at a higher density than a different target protein of a different exosome, wherein the different target protein comprises a conventional exosome protein or a variant thereof.

3. The exosome of claim 2, wherein the conventional exosome protein is selected from the group consisting of CD9, CD63, CD81, PDGFR, GPI anchor proteins, lactadherin, LAMP2, LAMP2B, and a fragment thereof.

4. The exosome of claim 1, wherein the target protein comprises a polypeptide of any of SEQ ID NOs: 3-15.

5. The exosome of claim 1, wherein the target protein comprises a polypeptide of SEQ ID NO: 14.

6. The exosome of claim 1, produced from a cell genetically modified to comprise the exogenous sequence, optionally wherein the cell is an HEK293 cell.

7. The exosome of claim 6, wherein the cell comprises a plasmid comprising the exogenous sequence.

8. The exosome of claim 6, wherein the cell comprises the exogenous sequence inserted into a genome of the cell.

9. The exosome of claim 8, wherein the exogenous sequence is inserted into a genomic site located 3′ or 5′ end of a genomic sequence encoding BASP1 or a fragment thereof.

10. The exosome of claim 8, wherein the exogenous sequence is inserted into a genomic sequence encoding BASP1.

11. The exosome of claim 1, wherein the target protein is a fusion protein comprising BASP1 or a fragment thereof, and a therapeutic peptide.

12. The exosome of claim 11, wherein the therapeutic peptide is selected from the group consisting of a natural peptide, a recombinant peptide, a synthetic peptide, or a linker to a therapeutic compound.

13. The exosome of claim 12, wherein the therapeutic compound is selected from the group consisting of nucleotides, amino acids, lipids, carbohydrates, and small molecules.

14. The exosome of claim 12 wherein the therapeutic peptide is an antibody or a fragment thereof.

15. The exosome of claim 12, wherein the therapeutic peptide is an antigen.

16. The exosome of claim 12, wherein the therapeutic peptide is a component of a genome editing complex.

17. The exosome of claim 16, wherein the genome editing complex is a CRISPR/Cas9 genome editing complex.

18. The exosome of claim 1, wherein the target protein is a fusion protein comprising BASP1 or a fragment thereof, and a viral capsid protein.

19. The exosome of claim 18, wherein the viral capsid protein is from adeno-associated virus

20. The exosome of claim 1, further comprising a second target protein, wherein the second target protein comprises PTGFRN, BSG, IGSF3, IGSF2, ITGB1, ITGA4, SLC3A2, ATP transporter, or a fragment thereof.


SEQUENCE LISTING (150 sequences)

SEQ ID NO: 1 - protein, 332 aa, Homo sapiens
MGAQFSKTAAKGEAAAERPGEAAVASSPSKANGQENGHVKVNGDASPAAAESGAKEELQANGSAPAADKEEPAAAGSGAASPSAAEKGEPAAAAAPEAGASPVEKEAPAEGEAAEPGSPTAAEGEAASAASSTSSPKAEDGATPSPSNETPKKKKKRFSFKKSFKLSGFSFKKNKKEAGEGGEAEAPAAEGGKDEAAGGAAAAAAEAGAASGEQAAAPGEEAAAGEEGAAGGDPQEAKPQEAAVAPEKPPASDETKAAEEPSKVEEKKAEEAGASAAACEAPSAAGPGAPPEQEAAPAEEPAAAAASSACAAPSQEAQPECSPEAPPAEAAE

SEQ ID NO: 2 - protein, 195 aa, Homo sapiens
MGSQSSKAPRGDVTAEEAAGASPAKANGQENGHVKSNGDLSPKGEGESPPVNGTDEAAGATGDAIEPAPPSQGAEAKGEVPPKETPKKKKKFSFKKPFKLSGLSFKRNRKEGGGDSSASSPTEEEQEQGEIGACSDEGTAQEGKAAATPESQEPQAKGAEASAASEEEAGPQATEPSTPSGPESGPTPASAEQNE

SEQ ID NO: 3 - protein, 227 aa, Homo sapiens
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 4 - protein, 30 aa, Artificial Sequence
  Description of Artificial Sequence Synthetic peptide
MGGKLSKKKKGYNVNDEKAKEKDKKAEGAA

SEQ ID NO: 5 - protein, 27 aa, Artificial Sequence
  Description of Artificial Sequence Synthetic peptide
MGGKLSKKKKGYNVNDEKAKEKDKKAE

SEQ ID NO: 6 - protein, 24 aa, Artificial Sequence
  Description of Artificial Sequence Synthetic peptide
MGGKLSKKKKGYNVNDEKAKEKDK

SEQ ID NO: 7 - protein, 21 aa, Artificial Sequence
  Description of Artificial Sequence Synthetic peptide
MGGKLSKKKKGYNVNDEKAKE

SEQ ID NO: 8 - protein, 18 aa, Artificial Sequence
  Description of Artificial Sequence Synthetic peptide
MGGKLSKKKKGYNVNDEK

SEQ ID NO: 9 - protein, 15 aa, Artificial Sequence
  Description of Artificial Sequence Synthetic peptide
MGGKLSKKKKGYNVN

SEQ ID NO: 10 - protein, 12 aa, Artificial Sequence
  Description of Artificial Sequence Synthetic peptide
MGGKLSKKKKGY

SEQ ID NO: 11 - protein, 11 aa, Artificial Sequence
  Description of Artificial Sequence Synthetic peptide
MGGKLSKKKKG

SEQ ID NO: 12 - protein, 10 aa, Artificial Sequence
  Description of Artificial Sequence Synthetic peptide
MGGKLSKKKK

SEQ ID NO: 13 - protein, 9 aa, Artificial Sequence
  Description of Artificial Sequence Synthetic peptide
MGGKLSKKK

SEQ ID NO: 14 - protein, 8 aa, Artificial Sequence
  Description of Artificial Sequence Synthetic peptide
MGGKLSKK

SEQ ID NO: 15 - protein, 7 aa, Artificial Sequence
  Description of Artificial Sequence Synthetic peptide
MGGKLSK

SEQ ID NO: 16 - protein, 8 aa, Artificial Sequence
  Description of Artificial Sequence Synthetic peptide
MGGKLAKK

SEQ ID NO: 17 - protein, 8 aa, Artificial Sequence
  Description of Artificial Sequence Synthetic peptide
MGGKFSKK

SEQ ID NO: 18 - protein, 8 aa, Artificial Sequence
  Description of Artificial Sequence Synthetic peptide
MGGKFAKK

SEQ ID NO: 19 - protein, 8 aa, Artificial Sequence
  Description of Artificial Sequence Synthetic peptide
MGGKSSKK

SEQ ID NO: 20 - protein, 8 aa, Artificial Sequence
  Description of Artificial Sequence Synthetic peptide
MGGKSAKK

SEQ ID NO: 21 - protein, 8 aa, Artificial Sequence
  Description of Artificial Sequence Synthetic peptide
MGGKQSKK

SEQ ID NO: 22 - protein, 8 aa, Artificial Sequence
  Description of Artificial Sequence Synthetic peptide
MGGKQAKK

SEQ ID NO: 23 - protein, 8 aa, Artificial Sequence
  Description of Artificial Sequence Synthetic peptide
MGGQLSKK

SEQ ID NO: 24 - protein, 8 aa, Artificial Sequence
  Description of Artificial Sequence Synthetic peptide
MGGQLAKK

SEQ ID NO: 25 - protein, 8 aa, Artificial Sequence
  Description of Artificial Sequence Synthetic peptide
MGGQFSKK

SEQ ID NO: 26 - protein, 8 aa, Artificial Sequence
  Description of Artificial Sequence Synthetic peptide
MGGQFAKK

SEQ ID NO: 27 - protein, 8 aa, Artificial Sequence
  Description of Artificial Sequence Synthetic peptide
MGGQSSKK

SEQ ID NO: 28 - protein, 8 aa, Artificial Sequence
  Description of Artificial Sequence Synthetic peptide
MGGQSAKK

SEQ ID NO: 29 - protein, 8 aa, Artificial Sequence
  Description of Artificial Sequence Synthetic peptide
MGGQQSKK

SEQ ID NO: 30 - protein, 8 aa, Artificial Sequence
  Description of Artificial Sequence Synthetic peptide
MGGQQAKK

SEQ ID NO: 31 - protein, 8 aa, Artificial Sequence
  Description of Artificial Sequence Synthetic peptide
MGAKLSKK

SEQ ID NO: 32 - protein, 8 aa, Artificial Sequence
  Description of Artificial Sequence Synthetic peptide
MGAKLAKK

SEQ ID NO: 33 - protein, 8 aa, Artificial Sequence
  Description of Artificial Sequence Synthetic peptide
MGAKFSKK

SEQ ID NO: 34 - protein, 8 aa, Artificial Sequence
  Description of Artificial Sequence Synthetic peptide
MGAKFAKK

SEQ ID NO: 35 - protein, 8 aa, Artificial Sequence
  Description of Artificial Sequence Synthetic peptide
MGAKSSKK

SEQ ID NO: 36 - protein, 8 aa, Artificial Sequence
  Description of Artificial Sequence Synthetic peptide
MGAKSAKK

SEQ ID NO: 37 - protein, 8 aa, Artificial Sequence
  Description of Artificial Sequence Synthetic peptide
MGAKQSKK

SEQ ID NO: 38 - protein, 8 aa, Artificial Sequence
  Description of Artificial Sequence Synthetic peptide
MGAKQAKK

SEQ ID NO: 39 - protein, 8 aa, Artificial Sequence
  Description of Artificial Sequence Synthetic peptide
MGAQLSKK

SEQ ID NO: 40 - protein, 8 aa, Artificial Sequence
  Description of Artificial Sequence Synthetic peptide
MGAQLAKK

SEQ ID NO: 41 - protein, 8 aa, Artificial Sequence
  Description of Artificial Sequence Synthetic peptide
MGAQFSKK

SEQ ID NO: 42 - protein, 8 aa, Artificial Sequence
  Description of Artificial Sequence Synthetic peptide
MGAQFAKK

SEQ ID NO: 43 - protein, 8 aa, Artificial Sequence
  Description of Artificial Sequence Synthetic peptide
MGAQSSKK

SEQ ID NO: 44 - protein, 8 aa, Artificial Sequence
  Description of Artificial Sequence Synthetic peptide
MGAQSAKK

SEQ ID NO: 45 - protein, 8 aa, Artificial Sequence
  Description of Artificial Sequence Synthetic peptide
MGAQQSKK

SEQ ID NO: 46 - protein, 8 aa, Artificial Sequence
  Description of Artificial Sequence Synthetic peptide
MGAQQAKK

SEQ ID NO: 47 - protein, 8 aa, Artificial Sequence
  Description of Artificial Sequence Synthetic peptide
MGSKLSKK

SEQ ID NO: 48 - protein, 8 aa, Artificial Sequence
  Description of Artificial Sequence Synthetic peptide
MGSKLAKK

SEQ ID NO: 49 - protein, 8 aa, Artificial Sequence
  Description of Artificial Sequence Synthetic peptide
MGSKFSKK

SEQ ID NO: 50 - protein, 8 aa, Artificial Sequence
  Description of Artificial Sequence Synthetic peptide
MGSKFAKK

SEQ ID NO: 51 - protein, 8 aa, Artificial Sequence
  Description of Artificial Sequence Synthetic peptide
MGSKSSKK

SEQ ID NO: 52 - protein, 8 aa, Artificial Sequence
  Description of Artificial Sequence Synthetic peptide
MGSKSAKK

SEQ ID NO: 53 - protein, 8 aa, Artificial Sequence
  Description of Artificial Sequence Synthetic peptide
MGSKQSKK

SEQ ID NO: 54 - protein, 8 aa, Artificial Sequence
  Description of Artificial Sequence Synthetic peptide
MGSKQAKK

SEQ ID NO: 55 - protein, 8 aa, Artificial Sequence
  Description of Artificial Sequence Synthetic peptide
MGSQLSKK

SEQ ID NO: 56 - protein, 8 aa, Artificial Sequence
  Description of Artificial Sequence Synthetic peptide
MGSQLAKK

SEQ ID NO: 57 - protein, 8 aa, Artificial Sequence
  Description of Artificial Sequence Synthetic peptide
MGSQFSKK

SEQ ID NO: 58 - protein, 8 aa, Artificial Sequence
  Description of Artificial Sequence Synthetic peptide
MGSQFAKK

SEQ ID NO: 59 - protein, 8 aa, Artificial Sequence
  Description of Artificial Sequence Synthetic peptide
MGSQSSKK

SEQ ID NO: 60 - protein, 8 aa, Artificial Sequence
  Description of Artificial Sequence Synthetic peptide
MGSQSAKK

SEQ ID NO: 61 - protein, 8 aa, Artificial Sequence
  Description of Artificial Sequence Synthetic peptide
MGSQQSKK

SEQ ID NO: 62 - protein, 8 aa, Artificial Sequence
  Description of Artificial Sequence Synthetic peptide
MGSQQAKK

SEQ ID NO: 63 - protein, 7 aa, Artificial Sequence
  Description of Artificial Sequence Synthetic peptide
MGGKLAK

SEQ ID NO: 64 - protein, 7 aa, Artificial Sequence
  Description of Artificial Sequence Synthetic peptide
MGGKFSK

SEQ ID NO: 65 - protein, 7 aa, Artificial Sequence
  Description of Artificial Sequence Synthetic peptide
MGGKFAK

SEQ ID NO: 66 - protein, 7 aa, Artificial Sequence
  Description of Artificial Sequence Synthetic peptide
MGGKSSK

SEQ ID NO: 67 - protein, 7 aa, Artificial Sequence
  Description of Artificial Sequence Synthetic peptide
MGGKSAK

SEQ ID NO: 68 - protein, 7 aa, Artificial Sequence
  Description of Artificial Sequence Synthetic peptide
MGGKQSK

SEQ ID NO: 69 - protein, 7 aa, Artificial Sequence
  Description of Artificial Sequence Synthetic peptide
MGGKQAK

SEQ ID NO: 70 - protein, 7 aa, Artificial Sequence
  Description of Artificial Sequence Synthetic peptide
MGGQLSK

SEQ ID NO: 71 - protein, 7 aa, Artificial Sequence
  Description of Artificial Sequence Synthetic peptide
MGGQLAK

SEQ ID NO: 72 - protein, 7 aa, Artificial Sequence
  Description of Artificial Sequence Synthetic peptide
MGGQFSK

SEQ ID NO: 73 - protein, 7 aa, Artificial Sequence
  Description of Artificial Sequence Synthetic peptide
MGGQFAK

SEQ ID NO: 74 - protein, 7 aa, Artificial Sequence
  Description of Artificial Sequence Synthetic peptide
MGGQSSK

SEQ ID NO: 75 - protein, 7 aa, Artificial Sequence
  Description of Artificial Sequence Synthetic peptide
MGGQSAK

SEQ ID NO: 76 - protein, 7 aa, Artificial Sequence
  Description of Artificial Sequence Synthetic peptide
MGGQQSK

SEQ ID NO: 77 - protein, 7 aa, Artificial Sequence
  Description of Artificial Sequence Synthetic peptide
MGGQQAK

SEQ ID NO: 78 - protein, 7 aa, Artificial Sequence
  Description of Artificial Sequence Synthetic peptide
MGAKLSK

SEQ ID NO: 79 - protein, 7 aa, Artificial Sequence
  Description of Artificial Sequence Synthetic peptide
MGAKLAK

SEQ ID NO: 80 - protein, 7 aa, Artificial Sequence
  Description of Artificial Sequence Synthetic peptide
MGAKFSK

SEQ ID NO: 81 - protein, 7 aa, Artificial Sequence
  Description of Artificial Sequence Synthetic peptide
MGAKFAK

SEQ ID NO: 82 - protein, 7 aa, Artificial Sequence
  Description of Artificial Sequence Synthetic peptide
MGAKSSK

SEQ ID NO: 83 - protein, 7 aa, Artificial Sequence
  Description of Artificial Sequence Synthetic peptide
MGAKSAK

SEQ ID NO: 84 - protein, 7 aa, Artificial Sequence
  Description of Artificial Sequence Synthetic peptide
MGAKQSK

SEQ ID NO: 85 - protein, 7 aa, Artificial Sequence
  Description of Artificial Sequence Synthetic peptide
MGAKQAK

SEQ ID NO: 86 - protein, 7 aa, Artificial Sequence
  Description of Artificial Sequence Synthetic peptide
MGAQLSK

SEQ ID NO: 87 - protein, 7 aa, Artificial Sequence
  Description of Artificial Sequence Synthetic peptide
MGAQLAK

SEQ ID NO: 88 - protein, 7 aa, Artificial Sequence
  Description of Artificial Sequence Synthetic peptide
MGAQFSK

SEQ ID NO: 89 - protein, 7 aa, Artificial Sequence
  Description of Artificial Sequence Synthetic peptide
MGAQFAK

SEQ ID NO: 90 - protein, 7 aa, Artificial Sequence
  Description of Artificial Sequence Synthetic peptide
MGAQSSK

SEQ ID NO: 91 - protein, 7 aa, Artificial Sequence
  Description of Artificial Sequence Synthetic peptide
MGAQSAK

SEQ ID NO: 92 - protein, 7 aa, Artificial Sequence
  Description of Artificial Sequence Synthetic peptide
MGAQQSK

SEQ ID NO: 93 - protein, 7 aa, Artificial Sequence
  Description of Artificial Sequence Synthetic peptide
MGAQQAK

SEQ ID NO: 94 - protein, 7 aa, Artificial Sequence
  Description of Artificial Sequence Synthetic peptide
MGSKLSK

SEQ ID NO: 95 - protein, 7 aa, Artificial Sequence
  Description of Artificial Sequence Synthetic peptide
MGSKLAK

SEQ ID NO: 96 - protein, 7 aa, Artificial Sequence
  Description of Artificial Sequence Synthetic peptide
MGSKFSK

SEQ ID NO: 97 - protein, 7 aa, Artificial Sequence
  Description of Artificial Sequence Synthetic peptide
MGSKFAK

SEQ ID NO: 98 - protein, 7 aa, Artificial Sequence
  Description of Artificial Sequence Synthetic peptide
MGSKSSK

SEQ ID NO: 99 - protein, 7 aa, Artificial Sequence
  Description of Artificial Sequence Synthetic peptide
MGSKSAK

SEQ ID NO: 100 - protein, 7 aa, Artificial Sequence
  Description of Artificial Sequence Synthetic peptide
MGSKQSK

SEQ ID NO: 101 - protein, 7 aa, Artificial Sequence
  Description of Artificial Sequence Synthetic peptide
MGSKQAK

SEQ ID NO: 102 - protein, 7 aa, Artificial Sequence
  Description of Artificial Sequence Synthetic peptide
MGSQLSK

SEQ ID NO: 103 - protein, 7 aa, Artificial Sequence
  Description of Artificial Sequence Synthetic peptide
MGSQLAK

SEQ ID NO: 104 - protein, 7 aa, Artificial Sequence
  Description of Artificial Sequence Synthetic peptide
MGSQFSK

SEQ ID NO: 105 - protein, 7 aa, Artificial Sequence
  Description of Artificial Sequence Synthetic peptide
MGSQFAK

SEQ ID NO: 106 - protein, 7 aa, Artificial Sequence
  Description of Artificial Sequence Synthetic peptide
MGSQSSK

SEQ ID NO: 107 - protein, 7 aa, Artificial Sequence
  Description of Artificial Sequence Synthetic peptide
MGSQSAK

SEQ ID NO: 108 - protein, 7 aa, Artificial Sequence
  Description of Artificial Sequence Synthetic peptide
MGSQQSK

SEQ ID NO: 109 - protein, 7 aa, Artificial Sequence
  Description of Artificial Sequence Synthetic peptide
MGSQQAK

SEQ ID NO: 110 - protein, 9 aa, Artificial Sequence
  Description of Artificial Sequence Synthetic peptide
MGAKLSKKK

SEQ ID NO: 111 - protein, 167 aa, Artificial Sequence
  Description of Artificial Sequence Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 112 - protein, 167 aa, Artificial Sequence
  Description of Artificial Sequence Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 113 - protein, 296 aa, Artificial Sequence
  Description of Artificial Sequence Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 114 - protein, 296 aa, Artificial Sequence
  Description of Artificial Sequence Synthetic polypeptide
MGGKLSKKKKGYNVNDEKAKEKDKKAEGAASGGSGGSDYKDDDDKGGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIYGSGGSGGSGGSGMASNFTQFVLVDNGGTGDVTVAPSNFANGIAEWISSNSRSQAYKVTCSVRQSSAQKRKYTIKVEVPKGAWRSYLNMELTIPIFATNSDCELIVKAMQGLLKDGNPIPSAIAANSGIY

SEQ ID NO: 115 - RNA, 680 nt, Artificial Sequence
  Description of Artificial Sequence Synthetic polynucleotide
augaagcccaccgagaacaacgaagacuucaacaucguggccguggccagcaacuucgcgaccacggaucucgaugcugaccgcgggaaguugcccggcaagaagcugccgcuggaggugcucaaagaguuggaagccaaugcccggaaagcuggcugcaccaggggcugucugaucugccugucccacaucaagugcacgcccaagaugaagaaguucaucccaggacgcugccacaccuacgaaggcgacaaagaguccgcacagggcggcauaggcgaggcgaucgucgacauuccugagauuccuggguucaaggacuuggagcccuuggagcaguucaucgcacaggucgaucuguguguggacugcacaacuggcugccucaaagggcuugccaacgugcaguguucugaccugcucaagaaguggcugccgcaacgcugugcgaccuuugccagcaagauccagggccagguggacaagaucaagggggccgguggugacuaaggauccaucgauaagcuucaucgaaacaugaggaucacccauaucugcagucgacaucgaaacaugaggaucacccaugucugcagucgacaucgaaacaugaggaucacccaugucugcagucgacaucgaaacaugaggaucacccaugucugcagucgacaucgaaa

SEQ ID NO: 116 - protein, 9 aa, Artificial Sequence
  Description of Artificial Sequence Synthetic peptide
  MOD_RES (3)..(3): Any amino acid
MGXKLSKKK

SEQ ID NO: 117 - protein, 9 aa, Artificial Sequence
  Description of Artificial Sequence Synthetic peptide
  MOD_RES (3)..(3): Any amino acid
  See specification as filed for detailed description of substitutions and preferred embodiments
MGXKLSKKK

SEQ ID NO: 118 - protein, 8 aa, Artificial Sequence
  Description of Artificial Sequence Synthetic peptide
  MOD_RES (3)..(3): Gly, Ala, or Ser
  MOD_RES (4)..(4): Lys or Gln
  MOD_RES (5)..(5): Leu, Phe, Ser, or Gln
  MOD_RES (6)..(6): Ser or Ala
  See specification as filed for detailed description of substitutions and preferred embodiments
MGXXXXKK

SEQ ID NO: 119 - DNA, 20 bp, Artificial Sequence
  Description of Artificial Sequence Synthetic primer
tggaggtgctcaaagagttg

SEQ ID NO: 120 - DNA, 17 bp, Artificial Sequence
  Description of Artificial Sequence Synthetic primer
ttgggcgtgcacttgat

SEQ ID NO: 121 - DNA, 13 bp, Artificial Sequence
  Description of Artificial Sequence Synthetic probe
gggcattggcttc

SEQ ID NO: 122 - protein, 60 aa, Artificial Sequence
  Description of Artificial Sequence Synthetic polypeptide
MGGKLSKKKKGYNVNDEKAKEKDKKAEGAASAGGGGSDYKDDDDKGGGGSVSKGEELFTG

SEQ ID NO: 123 - protein, 60 aa, Artificial Sequence
  Description of Artificial Sequence Synthetic polypeptide
MAGKLSKKKKGYNVNDEKAKEKDKKAEGAASAGGGGSDYKDDDDKGGGGSVSKGEELFTG

SEQ ID NO: 124 - protein, 60 aa, Artificial Sequence
  Description of Artificial Sequence Synthetic polypeptide
MGAKLSKKKKGYNVNDEKAKEKDKKAEGAASAGGGGSDYKDDDDKGGGGSVSKGEELFTG

SEQ ID NO: 125 - protein, 60 aa, Artificial Sequence
  Description of Artificial Sequence Synthetic polypeptide
MAAKLSKKKKGYNVNDEKAKEKDKKAEGAASAGGGGSDYKDDDDKGGGGSVSKGEELFTG

SEQ ID NO: 126 - protein, 57 aa, Artificial Sequence
  Description of Artificial Sequence Synthetic polypeptide
MGGKLSKKKKGYNVNDEKAKEKDKKAESAGGGGSDYKDDDDKGGGGSVSKGEELFTG

SEQ ID NO: 127 - protein, 54 aa, Artificial Sequence
  Description of Artificial Sequence Synthetic polypeptide
MGGKLSKKKKGYNVNDEKAKEKDKSAGGGGSDYKDDDDKGGGGSVSKGEELFTG

SEQ ID NO: 128 - protein, 51 aa, Artificial Sequence
  Description of Artificial Sequence Synthetic polypeptide
MGGKLSKKKKGYNVNDEKAKESAGGGGSDYKDDDDKGGGGSVSKGEELFTG

SEQ ID NO: 129 - protein, 48 aa, Artificial Sequence
  Description of Artificial Sequence Synthetic polypeptide
MGGKLSKKKKGYNVNDEKSAGGGGSDYKDDDDKGGGGSVSKGEELFTG

SEQ ID NO: 130 - protein, 45 aa, Artificial Sequence
  Description of Artificial Sequence Synthetic polypeptide
MGGKLSKKKKGYNVNSAGGGGSDYKDDDDKGGGGSVSKGEELFTG

SEQ ID NO: 131 - protein, 42 aa, Artificial Sequence
  Description of Artificial Sequence Synthetic polypeptide
MGGKLSKKKKGYSAGGGGSDYKDDDDKGGGGSVSKGEELFTG

SEQ ID NO: 132 - protein, 39 aa, Artificial Sequence
  Description of Artificial Sequence Synthetic polypeptide
MGGKLSKKKSAGGGGSDYKDDDDKGGGGSVSKGEELFTG

SEQ ID NO: 133 - protein, 36 aa, Artificial Sequence
  Description of Artificial Sequence Synthetic polypeptide
MGGKLSSAGGGGSDYKDDDDKGGGGSVSKGEELFTG

SEQ ID NO: 134 - protein, 33 aa, Artificial Sequence
  Description of Artificial Sequence Synthetic polypeptide
MGGSAGGGGSDYKDDDDKGGGGSVSKGEELFTG

SEQ ID NO: 135 - protein, 54 aa, Artificial Sequence
  Description of Artificial Sequence Synthetic polypeptide
MGGKLSKKKKGYNVNDEKAKEKDKKAEGAASAGGGGSDYKDDDDKGGGGSVSKG

SEQ ID NO: 136 - protein, 36 aa, Artificial Sequence
  Description of Artificial Sequence Synthetic polypeptide
MGGKLSKKKKGYSAGGGGSDYKDDDDKGGGGSVSKG

SEQ ID NO: 137 - protein, 35 aa, Artificial Sequence
  Description of Artificial Sequence Synthetic polypeptide
MGGKLSKKKKGSAGGGGSDYKDDDDKGGGGSVSKG

SEQ ID NO: 138 - protein, 34 aa, Artificial Sequence
  Description of Artificial Sequence Synthetic polypeptide
MGGKLSKKKKSAGGGGSDYKDDDDKGGGGSVSKG

SEQ ID NO: 139 - protein, 33 aa, Artificial Sequence
  Description of Artificial Sequence Synthetic polypeptide
MGGKLSKKKSAGGGGSDYKDDDDKGGGGSVSKG

SEQ ID NO: 140 - protein, 32 aa, Artificial Sequence
  Description of Artificial Sequence Synthetic polypeptide
MGGKLSKKSAGGGGSDYKDDDDKGGGGSVSKG

SEQ ID NO: 141 - protein, 31 aa, Artificial Sequence
  Description of Artificial Sequence Synthetic polypeptide
MGGKLSKSAGGGGSDYKDDDDKGGGGSVSKG

SEQ ID NO: 142 - protein, 30 aa, Artificial Sequence
  Description of Artificial Sequence Synthetic polypeptide
MGGKLSSAGGGGSDYKDDDDKGGGGSVSKG

SEQ ID NO: 143 - protein, 30 aa, Artificial Sequence
  Description of Artificial Sequence Synthetic polypeptide
MGGKLDKKKKGYNVNDEKAKEKDKKAEGAA

SEQ ID NO: 144 - protein, 30 aa, Artificial Sequence
  Description of Artificial Sequence Synthetic polypeptide
MGGKLAKKKKGYNVNDEKAKEKDKKAEGAA

SEQ ID NO: 145 - protein, 30 aa, Artificial Sequence
  Description of Artificial Sequence Synthetic polypeptide
MGGKQSKKKKGYNVNDEKAKEKDKKAEGAA

SEQ ID NO: 146 - protein, 30 aa, Artificial Sequence
  Description of Artificial Sequence Synthetic polypeptide
MGAKKKKKRFSFKKSFKLSGFSFKKNKKEA

SEQ ID NO: 147 - protein, 30 aa, Artificial Sequence
  Description of Artificial Sequence Synthetic polypeptide
MAAKKKKKRFSFKKSFKLSGFSFKKNKKEA

SEQ ID NO: 148 - protein, 30 aa, Artificial Sequence
  Description of Artificial Sequence Synthetic polypeptide
MGAKKSKKRFSFKKSFKLSGFSFKKNKKEA

SEQ ID NO: 149 - protein, 30 aa, Artificial Sequence
  Description of Artificial Sequence Synthetic polypeptide
MGAKKAKKRFSFKKPFKLSGFSFKKNKKEA

SEQ ID NO: 150 - protein, 153 aa, Artificial Sequence
  Description of Artificial Sequence Synthetic polypeptide
MGGKLSKKKKSAGGSGGSTSGSGDYKDDDDKGSGFEMDQVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSS